(19) United States
(12) Patent Application Publication
(10) Pub. No.: US 2022/0381783 A1
(43) Pub. Date: Dec. 1, 2022

(54) BIOMARKER

(71) Applicant: Oxford University Innovation Limited, Oxford (GB)

(72) Inventors: Kristijan RAMADAN, Oxford (GB); John FIELDEN, Oxford (GB); Tim MAUGHAN, Oxford (GB)

(21) Appl. No.: 17/632,014

(22) PCT Filed: Jul. 30, 2020
(86) PCT No.: PCT/GB2020/051836

§ 371 Date: Feb. 1, 2022

(30) Foreign Application Priority Data
Aug. 2, 2019 (GB) 1911078.2

(51) Int. Cl. G01N 33/574

(52) U.S. Cl. CPC G01N 33/57419; G01N 2333/99; G01N 2800/52

(57) ABSTRACT

The invention relates to a method for identifying a cancer that is predicted to respond to treatment with a topoisomerase 1 (TOP1) inhibitor. The invention also extends to a method of treating cancer in a subject and a method of selecting a cancer patient for treatment with a cancer therapy. The invention further extends to use of cancer cells, such as primary colon cancer cells, as a biomarker for a patients response to treatment (insensitivity or sensitivity) with a particular chemotherapeutic agent, such as a TOP1 inhibitor.

### CROSS-REFERENCE TO RELATED APPLICATIONS

This application is the National Stage of International Application No. PCT/GB2020/051836, filed Jul. 30, 2020, which claims priority to GB 1911078.2, filed Aug. 2, 2019, which are entirely incorporated herein by reference.

### SEQUENCE LISTING

This application contains a sequence listing filed in electronic form as an ASCII.txt file entitled “JA100287P.WOP_Sequence listing_ST25.TXT”, created on Jul. 19, 2022 and having a size of 22 kb. The content of the sequence listing is incorporated herein in its entirety.

The present invention relates to a method for identifying a cancer that is predicted to respond to a chemotherapeutic agent. In particular, to a method for identifying a cancer that is predicted to respond to treatment with a topoisomerase 1 (TOP1) inhibitor, such as irinotecan. The cancer may be metastatic colorectal cancer. The invention also extends to a method of treating cancer in a subject and a method of selecting a cancer patient for treatment with a cancer therapy. The invention further extends to use of cancer cells, such as primary colon cancer cells, as a biomarker for a patient's response to treatment (insensitivity or sensitivity) with a particular chemotherapeutic agent, such as a TOP1 inhibitor.

Topoisomerase 1 (TOP1) resolves DNA topological stress accumulated during DNA replication and transcription. As part of its catalytic cycle, TOP1 forms an intermediate which is covalently bound to DNA, known as a TOP1 cleavage complex (TOP1cc). TOP1ccs are usually transient but can become trapped if TOP1 cleaves near a DNA alteration or is exposed to TOP1 inhibitors. Owing to their bulky nature, TOP1ccs hinder the progression of DNA replication and transcription, and are therefore highly cytotoxic. This is demonstrated by the effectiveness of the widely used class of anti-cancer drugs, known as TOP1 inhibitors, which stabilise TOP1ccs by binding the TOP1-DNA interface. TOP1 inhibitors are routinely used to treat cancer, including ovarian, colon, and lung cancers. In fact, up to 50% of all chemotherapeutic regimens consist of treatment with topoisomerases inhibitors, such as irinotecan (a camptothecin (CPT) derivate). However, resistance to TOP1 inhibitors is common. This underscores the need to identify molecular biomarkers and determinants of resistance to improve patient stratification and outcomes.

The lack of good biomarkers to indicate which patients will respond to a particular drug, for example a TOP1 inhibitor, is a hindrance for cancer treatment. In particular, there is an unmet need for novel biomarkers that have value to facilitate the implementation of more targeted therapeutic strategies in cancer patients that could improve overall clinical outcomes.

It is therefore an aim of the present invention to provide a biomarker that may be used to identify cancer patients that will and will not respond to particular treatments, for example to a TOP1 inhibitor, such as irinotecan. This will allow patients to be given the most appropriate treatment quickly and avoid administering treatment which will not be effective and/or has an undesirable side effect. Not only are there benefits to the patient, but it is clear that there will also be significant cost savings in identifying and selecting therapy-responsive patients. The invention is based on a simple and user friendly method that can be routinely performed in any pathology lab, in particular it may be performed on a cancer patient biopsy sample of surgically resected cancer.

The inventors of the present invention have surprisingly found that the level of expression of the SPRTN enzyme and/or the level of expression of the SPRTN gene in cancer cells strongly correlates with the clinical resistance or sensitivity of the cancer cells to particular chemotherapeutic agents. In particular the level of expression of the SPRTN enzyme and/or the SPRTN gene in primary colorectal cancer cells in a patient correlates with the response of a metastatic colorectal cancer to treatment with a TOP1 inhibitor, such as the CPT-derivate irinotecan. Specifically, patients with high levels of expression of the SPRTN enzyme and/or the SPRTN gene in primary colorectal cancer cells do not respond well to irinotecan therapy, their metastases do not regress and typically the patients die.

The level of expression of the SPRTN enzyme in a population of cells, such as cancer cells, may be determined by a histological (H)-index. This is an arbitrary indicator that measures i) area (as a % of the total area) of the cancer sample which expresses the SPRTN enzyme and/or gene, and ii) the intensity of the protein or mRNA signal when it is observed. The expression of SPRTN enzyme may be analysed by using a SPRTN-specific antibody. The intensity of SPRTN protein signal is arbitrary and is usually defined by a well-trained pathologist as 0=no signal, 1=weak signal, 2=moderate signal and 3=strong signal. The H-index is=(% of area affected)×(intensity), that is % of total area of a cancer cells in a sample that expresses the SPRTN enzyme times the intensity of the SPRTN enzyme signal when it is detected. Preferably the H-index is determined by considering only cancer cells in a sample. The skilled pathologists can readily determine which cells in a sample are cancer cells based on the morphology of the cells. The H-index may therefore be calculated as (the percentage of cancers cells in a sample that express the SPRTN enzyme) X (the intensity of SPRTN expression in those cells). The H-index may be determined in a histopathology laboratory on a tissue sample using a labelled anti-SPRTN antibody and a microscope.

Therefore, according to one aspect of the invention, there is provided a method for identifying a cancer that is predicted to respond to treatment with a Topoisomerase 1 (TOP1) inhibitor, the method comprising:
i. obtaining a sample of cancer cells from a subject; and
ii. detecting the expression of SPRTN enzyme and/or SPRTN mRNA in the sample of cancer cells;
   wherein if the level of SPRTN enzyme and/or SPRTN mRNA in the sample is low then the subject is predicted to respond to treatment with a TOP1 inhibitor; or wherein if the level of SPRTN enzyme and/or SPRTN mRNA in the sample is high then the subject is predicted not to respond to treatment with a TOP1 inhibitor.

Preferably the invention provides a method for identifying a subject with a metastatic colorectal cancer that is predicted to respond to treatment with a Topoisomerase 1 (TOP1) inhibitor, the method comprising:
i. obtaining a sample of primary colorectal cancer cells from the subject; and
ii. detecting the expression of SPRTN enzyme and/or SPRTN mRNA in the sample of primary cancer cells;
   wherein if the level of SPRTN enzyme and/or SPRTN mRNA in the sample is low then the metastasis of the colorectal cancer is predicted to respond to treatment with a TOP1 inhibitor.

Obtaining a sample of primary colorectal cancer cells is routine in the diagnosis of colorectal cancer.

Typically if a subject with metastatic colorectal cancer responds to therapy they will survive.

According to another aspect of the invention, there is provided a method for identifying a subject with a metastatic colorectal cancer that is predicted not to respond to treatment with a Topoisomerase 1 (TOP1) inhibitor, the method comprising:
i. obtaining a sample of primary colorectal cancer cells from the subject; and
ii. detecting the expression of SPRTN enzyme and/or SPRTN mRNA in the sample of primary cancer cells;
   wherein if the level of SPRTN enzyme and/or SPRTN mRNA in the sample is high then the metastatic cancer is predicted not to respond to treatment with a TOP1 inhibitor.

A low level of SPRTN enzyme expression may be defined as that observed in a sample with an H-score of 100 or below.

A high level of SPRTN enzyme expression may be defined as that observed in a sample with an H-score of above 100.

The change in SPRTN enzyme expression may correlate with a change in the level of SPRTN mRNA.

Preferably, in a method of the invention, the subject has already been diagnosed with cancer. The subject may already have been diagnosed with colorectal cancer, this may include primary colorectal cancer and/or metastatic colorectal cancer.

Advantageously, the method of the invention allows subjects with cancer, and in particular those with metastatic colorectal cancer, to be stratified into those which are expected to respond to therapy with a TOP1 inhibitor and those that are predicted not to respond to therapy with a TOP1 inhibitor or those predicted to respond poorly to therapy with a TOP1 inhibitor.

The method of the invention may further comprise a step (iii) of predicting that the subject, in particular a metastatic colorectal cancer patient, will respond to treatment with a TOP1 inhibitor if the level of SPRTN enzyme and/or SPRTN mRNA in the cancer cells sample is low (H-index low).

The method of the invention may further comprise a step (iii) of predicting that the subject, in particular a metastatic colorectal cancer patient, will not respond to treatment with a TOP1 inhibitor if the level of SPRTN protein and/or SPRTN mRNA in the cancer cells sample is high (H-index high).

According to another aspect of the invention, there is provided a kit for identifying a subject with a cancer that is predicted to respond to treatment with a TOP1 inhibitor, the kit comprising: detection means for detecting the SPRTN enzyme and/or SPRTN mRNA in a sample of cancer cells; and instructions that if the level of SPRTN enzyme and/or SPRTN mRNA in the sample is low, then the patient is predicted to respond to treatment with a TOP1 inhibitor, or that if the level of SPRTN enzyme and/or SPRTN mRNA in the sample is high, then the patient is predicted not to respond to treatment with a TOP1 inhibitor. The kit may be for use with a sample of primary or metastatic colorectal cancer cells. The cancer that is predicted to respond may be a metastatic colorectal cancer.

The sample may be a primary colorectal cancer biopsy or cells derived there form.

Preferably, the kit comprises one or more control or reference samples and a SPRTN specific antibody for use in the immunohistological staining of cells.

Alternatively or additionally, the kit may comprise one or more control or reference samples and primers specific for SPRTN mRNA for use in detecting SPRTN gene expression.

The detection means is preferably configured to detect SPRTN enzyme or SPRTN mRNA in a sample.

The detection means may be an antibody to detect SPRTN enzyme. The skilled person would appreciate that the level SPRTN enzyme, visualised by immunohistochemistry using a specific SPRTN antibody, directly correlates with the response of the subject to TOP1-inhibitor therapy. The level of SPRTN enzyme may be determined in a primary tumour biopsy or in a metastatic tumour biopsy, and may correlate to the response of the primary tumour and/or the metastatic tumour to treatment with a TOP1 inhibitor.

The detection means may be a primer or probe to detect the SPRTN mRNA. PCR or RNA sequencing may be used to detect the level of SPRTN mRNA in a sample. The skilled person would appreciate that the level of mRNA in a sample is indicative of the level of gene expression in the sample. The skilled person would appreciate that the level of SPRTN mRNA, for example determined by PCR, directly correlates with the response of the subject to TOP1-inhibitor therapy.

TOP1 (Topoisomerase 1) is an enzyme that is responsible for releasing topological stress accumulated during the separation of DNA strands. It achieves this by cleaving a single strand of DNA, rearranging the cleaved strand and ligating it back together in order to release tension that has built up during DNA replication and/or RNA transcription. TOP1 activity may, therefore, be detected by performing an in vitro DNA cleavage assay. The TOP1 enzyme may be encoded by the gene, TOP1:

Homo sapiens DNA topoisomerase I (TOP1), mRNA
NCBI Reference Sequence: NM_003286.4
>NM_003286.4 Homo sapiens DNA topoisomerase I (TOP1), mRNA
[SEQ ID NO: 1]
CAAATGCGAACTTAGGCTGTTACACAACTGCTGGGGTCTGTTCTCGCCGCCCGCCCGGCAGTCAGGCAGC
GTCGCCGCCGTGGTAGCAGCCTCAGCCGTTTCTGGAGTCTCGGGCCCACAGTCACCGCCGCTTACCTGCG
CCTCCTCGAGCCTCCGGAGTCCCCGTCCGCCCGCACAGGCCGGTTCGCCGTCTGCGTCTCCCCCACGCCG
CCTCGCCTGCCGCCGCGCTCGTCCCTCCGGGCCGACATGAGTGGGGACCACCTCCACAACGATTCCCAGA
TCGAAGCGGATTTCCGATTGAATGATTCTCATAAACACAAAGATAAACACAAAGATCGAGAACACCGGCA
CAAAGAACACAAGAAGGAGAAGGACCGGGAAAAGTCCAAGCATAGCAACAGTGAACATAAAGATTCTGAA
AAGAAACACAAAGAGAAGGAGAAGACCAAACACAAAGATGGAAGCTCAGAAAAGCATAAAGACAAACATA
AAGACAGAGACAAGGAAAAACGAAAAGAGGAAAAGGTTCGAGCCTCTGGGGATGCAAAAATAAAGAAGGA
GAAGGAAAATGGCTTCTCTAGTCCACCACAAATTAAAGATGAACCTGAAGATGATGGCTATTTTGTTCCT
CCTAAAGAGGATATAAAGCCATTAAAGAGACCTCGAGATGAGGATGATGCTGATTATAAACCTAAGAAAA
TTAAAACAGAAGATACCAAGAAGGAGAAGAAAAGAAAACTAGAAGAAGAAGAGGATGGTAAATTGAAAAA
ACCCAAGAATAAAGATAAAGATAAAAAAGTTCCTGAGCCAGATAACAAGAAAAAGAAGCCGAAGAAAGAA
GAGGAACAGAAGTGGAAATGGTGGGAAGAAGAGCGCTATCCTGAAGGCATCAAGTGGAAATTCCTAGAAC
ATAAAGGTCCAGTATTTGCCCCACCATATGAGCCTCTTCCAGAGAATGTCAAGTTTTATTATGATGGTAA
AGTCATGAAGCTGAGCCCCAAAGCAGAGGAAGTAGCTACGTTCTTTGCAAAAATGCTCGACCATGAATAT
ACTACCAAGGAAATATTTAGGAAAAATTTCTTTAAAGACTGGAGAAAGGAAATGACTAATGAAGAGAAGA
ATATTATCACCAACCTAAGCAAATGTGATTTTACCCAGATGAGCCAGTATTTCAAAGCCCAGACGGAAGC
TCGGAAACAGATGAGCAAGGAAGAGAAACTGAAAATCAAAGAGGAGAATGAAAAATTACTGAAAGAATAT
GGATTCTGTATTATGGATAACCACAAAGAGAGGATTGCTAACTTCAAGATAGAGCCTCCTGGACTTTTCC
GTGGCCGCGGCAACCACCCCAAGATGGGCATGCTGAAGAGACGAATCATGCCCGAGGATATAATCATCAA
CTGTAGCAAAGATGCCAAGGTTCCTTCTCCTCCTCCAGGACATAAGTGGAAAGAAGTCCGGCATGATAAC
AAGGTTACTTGGCTGGTTTCCTGGACAGAGAACATCCAAGGTTCCATTAAATACATCATGCTTAACCCTA
GTTCACGAATCAAGGGTGAGAAGGACTGGCAGAAATACGAGACTGCTCGGCGGCTGAAAAAATGTGTGGA
CAAGATCCGGAACCAGTATCGAGAAGACTGGAAGTCCAAAGAGATGAAAGTCCGGCAGAGAGCTGTAGCC
CTGTACTTCATCGACAAGCTTGCTCTGAGAGCAGGCAATGAAAAGGAGGAAGGAGAAACAGCGGACACTG
TGGGCTGCTGCTCACTTCGTGTGGAGCACATCAATCTACACCCAGAGTTGGATGGTCAGGAATATGTGGT
AGAGTTTGACTTCCTCGGGAAGGACTCCATCAGATACTATAACAAGGTCCCTGTTGAGAAACGAGTTTTT
AAGAACCTACAACTATTTATGGAGAACAAGCAGCCCGAGGATGATCTTTTTGATAGACTCAATACTGGTA
TTCTGAATAAGCATCTTCAGGATCTCATGGAGGGCTTGACAGCCAAGGTATTCCGTACATACAATGCCTC
CATCACGCTACAGCAGCAGCTAAAAGAACTGACAGCCCCGGATGAGAACATCCCAGCGAAGATCCTTTCT
TATAACCGTGCCAATCGAGCTGTTGCAATTCTTTGTAACCATCAGAGGGCACCACCAAAAACTTTTGAGA
AGTCTATGATGAACTTGCAAACTAAGATTGATGCCAAGAAGGAACAGCTAGCAGATGCCCGGAGAGACCT
GAAAAGTGCTAAGGCTGATGCCAAGGTCATGAAGGATGCAAAGACGAAGAAGGTAGTAGAGTCAAAGAAG
AAGGCTGTTCAGAGACTGGAGGAACAGTTGATGAAGCTGGAAGTTCAAGCCACAGACCGAGAGGAAAATA
AACAGATTGCCCTGGGAACCTCCAAACTCAATTATCTGGACCCTAGGATCACAGTGGCTTGGTGCAAGAA
GTGGGGTGTCCCAATTGAGAAGATTTACAACAAAACCCAGCGGGAGAAGTTTGCCTGGGCCATTGACATG
GCTGATGAAGACTATGAGTTTTAGCCAGTCTCAAGAGGCAGAGTTCTGTGAAGAGGAACAGTGTGGTTTG
GGAAAGATGGATAAACTGAGCCTCACTTGCCCTCGTGCCTGGGGGAGAGAGGCAGCAAGTCTTAACAAAC
CAACATCTTTGCGAAAAGATAAACCTGGAGATATTATAAGGGAGAGCTGAGCCAGTTGTCCTATGGACAA
CTTATTTAAAAATATTTCAGATATCAAAATTCTAGCTGTATGATTTGTTTTGAATTTTGTTTTTATTTTC
AAGAGGGCAAGTGGATGGGAATTTGTCAGCGTTCTACCAGGCAAATTCACTGTTTCACTGAAATGTTTGG
ATTCTCTTAGCTACTGTATGCAAAGTCCGATTATATTGGTGCGTTTTTACAGTTAGGGTTTTGCAATAAC
TTCTATATTTTAATAGAAATAAATTCCTAAACTCCCTTCCCTCTCTCCCATTTCAGGAATTTAAAATTAA
GTAGAACAAAAAACCCAGCGCACCTGTTAGAGTCGTCACTCTCTATTGTCATGGGGATCAATTTTCATTA
AACTTGAAGCAGTCGTGGCTTTGGCAGTGTTTTGGTTCAGACACCTGTTCACAGAAAAAGCATGATGGGA
AAATATTTCCTGACTTGAGTGTTCCTTTTTAAATGTGAATTTTTATTTCTTTTTAATTATTTTAAAATAT
TTAAACCTTTTTCTTGATCTTAAAGATCGTGTAGATTGGGGTTGGGGAGGGATGAAGGGCGAGTGAATCT
AAGGATAATGAAATAATCAGTGACTGAAACCATTTTCCCATCATCCTTTGTTCTGAGCATTCGCTGTACC
CTTTAAGATATCCATCTTTTTCTTTTTAACCCTAATCTTTCACTTGAAAGATTTTATTGTATAAAAAGTT
TCACAGGTCAATAAACTTAGAGGAAAATGAGTATTTGGTCCAAAAAAAGGAAAAATAATCAAGATTTTAG
GGCTTTTATTTTTTCTTTTGTAATTGTGTAAAAAATGGAAAAAAACATAAAAAGCAGAATTTTAATGTGA
AGACATTTTTTGCTATAATCATTAGTTTTAGAGGCATTGTTAGTTTAGTGTGTGTGCAGAGTCCATTTCC
CACATCTTTCCTCAAGTATCTTCTATTTTTATCATGAATTCCCTTTTAATCAACTGTAGGTTATTTAAAA
TAAATTCCTACAACTTAATGGAAA
Homo sapiens DNA topoisomerase I (TOP1) protein sequence
NCBI Reference Sequence: NM_003277.1
>NP_003277.1 DNA topoisomerase I [Homo sapiens]
[SEQ ID NO: 2]
MSGDHLHNDSQIEADFRLNDSHKHKDKHKDREHRHKEHKKEKDREKSKHSNSEHKDSEKKHKEKEKTKHK
DGSSEKHKDKHKDRDKEKRKEEKVRASGDAKIKKEKENGFSSPPQIKDEPEDDGYFVPPKEDIKPLKRPR
DEDDADYKPKKIKTEDTKKEKKRKLEEEEDGKLKKPKNKDKDKKVPEPDNKKKKPKKEEEQKWKWWEEER
YPEGIKWKFLEHKGPVFAPPYEPLPENVKFYYDGKVMKLSPKAEEVATFFAKMLDHEYTTKEIFRKNFFK
DWRKEMTNEEKNIITNLSKCDFTQMSQYFKAQTEARKQMSKEEKLKIKEENEKLLKEYGFCIMDNHKERI
ANFKIEPPGLFRGRGNHPKMGMLKRRIMPEDIIINCSKDAKVPSPPPGHKWKEVRHDNKVTWLVSWTENI
QGSIKYIMLNPSSRIKGEKDWQKYETARRLKKCVDKIRNQYREDWKSKEMKVRQRAVALYFIDKLALRAG
NEKEEGETADTVGCCSLRVEHINLHPELDGQEYWEFDFLGKDSIRYYNKVPVEKRVFKNLQLFMENKQP
EDDLFDRLNTGILNKHLQDLMEGLTAKVFRTYNASITLQQQLKELTAPDENIPAKILSYNRANRAVAILC
NHQRAPPKTFEKSMMNLQTKIDAKKEQLADARRDLKSAKADAKVMKDAKTKKVVESKKKAVQRLEEQLMK
LEVQATDREENKQIALGTSKLNYLDPRITVAWCKKWGVPIEKIYNKTQREKFAWAIDMADEDYEF

SPRTN is a gene that encodes the enzyme, SPRTN also known as SPARTAN, DVC1 or Clorf124. A nucleotide sequence that encodes one embodiment of the human SPRTN gene is referred to here:

Homo sapiens SprT-like N-terminak domain (SPRTN), transcript variant 1,
mRNA
NCBI Reference Sequence: NM_032018.7
[SEQ ID NO: 3]
AGTCCTGGTCCTCGGCTAGGCGGCTTGGGGTCGCGGCGTAACTGGGGAGCCAGCCTGACGCCGGCGGACC
CCGCCTGTGATCCTGGCAACGATGGATGATGACTTGATGTTGGCACTGCGGCTTCAGGAGGAGTGGAACT
TGCAGGAGGCGGAGCGCGATCATGCCCAGGAGTCCCTGTCGCTAGTGGACGCGTCGTGGGAGTTGGTGGA
CCCCACACCGGACTTGCAGGCACTGTTTGTTCAGTTTAACGACCAATTCTTCTGGGGCCAGCTGGAGGCC
GTCGAGGTGAAGTGGAGCGTGCGAATGACCCTGTGTGCTGGGATATGCAGCTATGAAGGGAAGGGTGGAA
TGTGTTCCATCCGTCTCAGCGAACCCCTTTTGAAGTTGAGGCCAAGAAAGGATCTTGTAGAGACCCTCCT
GCATGAAATGATACATGCCTATTTATTTGTCACTAATAACGACAAAGACCGAGAAGGGCATGGTCCAGAA
TTTTGTAAACATATGCATCGCATCAACAGCCTGACTGGAGCCAATATAACGGTATACCATACTTTTCACG
ATGAGGTGGATGAGTATCGGCGACACTGGTGGCGCTGCAATGGGCCGTGCCAGCACAGGCCACCGTATTA
CGGCTATGTCAAACGAGCTACTAACAGGGAACCCTCTGCTCATGACTATTGGTGGGCTGAGCACCAGAAA
ACCTGTGGAGGCACTTACATAAAAATCAAGGAACCAGAGAATTACTCAAAAAAAGGCAAAGGAAAGGCAA
AACTAGGAAAGGAACCAGTATTGGCCGCAGAGAATAAAGATAAACCCAACAGAGGTGAGGCCCAGCTAGT
AATCCCTTTTAGTGGGAAAGGATATGTTCTAGGAGAAACAAGCAATTTACCTTCACCTGGGAAACTGATC
ACTTCACATGCCATTAATAAAACCCAAGATCTTTTAAATCAAAACCATTCAGCAAATGCTGTAAGACCTA
ATTCTAAAATCAAGGTGAAATTTGAACAGAATGGTTCAAGTAAAAATTCTCATCTGGTCTCCCCTGCTGT
TAGTAACAGTCACCAAAATGTTCTAAGCAACTACTTTCCTAGAGTATCATTTGCCAACCAAAAGGCTTTC
AGAGGTGTGAATGGATCTCCAAGGATAAGTGTAACAGTTGGCAACATCCCTAAAAACTCAGTCTCTTCTA
GTTCTCAGAGAAGGGTTTCATCTTCTAAGATATCCCTAAGAAATTCTTCAAAAGTAACGGAATCAGCATC
TGTGATGCCATCCCAGGATGTGAGTGGGTCTGAAGATACATTCCCAAATAAACGACCTAGGCTAGAAGAT
AAGACTGTTTTTGACAATTTTTTTATCAAGAAAGAGCAAATAAAAAGCAGTGGTAATGATCCAAAGTATA
GTACAACCACAGCTCAGAATTCCAGCAGTTCATCCAGTCAGAGCAAAATGGTTAATTGCCCAGTTTGTCA
GAATGAAGTTCTGGAGTCTCAGATTAATGAGCACTTGGACTGGTGCCTTGAAGGTGACAGCATCAAAGTC
AAAAGCGAAGAAAGTCTTTGAAAAAGGTTTCAAAGTCTCAAGTACCACCTGTATTATCTCACTAATGTGC
TATGTCAGCCAGTCAGGAAGTTCTGGTTAATACTAAGATTTGTAGGTTATAATCTAGTTCACATAACCAA
TAGAAAGTGTCCTATTTTATATATACGCATATAAGATTGTAATTTTAAGATGTTTTGTGTCTCAGGGTGC
TACATTCACTCTTGCCTTAGGTATACTGTAACCCAGGTTCTGCCTGTCGTGTATAAGTTTTAGATACTTT
TGTTCTTTCTTGCTCTTAAGGATTTTAAAAACCTGTTAATCTTTTTATTTGTATACTTTCCTAAAAATAT
TCATATGGGGAATCCTGTCAGGTGTTTGGTTATATTGACTATTTATTAATAGTATTAGAACTCATTCCCT
GAACTGATGTAAATCTTCATAGTGTCAGACATACTGACCAAAACCACAATCTAGACTACAAAGTATATTG
TTTTAGAGTACTCAAATTGTATTATTTATTAATTTTTTTGTTTGCAAAATCTTAACAGGAACTGTATTTT
CTATATTTTAAAGAATTTTATTTGTCCCACTTTTACTAAACAGTGGCAGCAGATTTTAAGTTAAAGAATA
TGGAATATAGTAAAATAAGTAAATTTCTTTTGGAATATTTTTAGTAACAAATAGCCACTATAATTCTGTA
GGCCAAATTTTATATTGAGTTTAGCTGTTTTCTCAAAATTTAGCAGAGTGGTTAAAATTCTGTGCTGATA
AGTAACTGATACATATAACATAAACATAACAAAGTTGCCTAGTTGATGTAACAGTGGAAAGTTATCTGGA
AATAGTATTTTGAACTTTAAGCCAAGTTTAAAACATTATAATAAAAGGAATACCATTTGTGCATTTTAAG
TAATCTTTTTTAAAAAAAATATTTTCCATGTTATAGGGAAAGGACAAAGAGACTTTTATCAGTTTGCTTT
TTGTCTTGTGGCTGTACATGCTGTTGGCATAGCCCTAACACAGTTGTTCACAAGTTTTCTTTTTTCTTGT
TGCAATTTTCCTTCACTTTGTTGTAATACAGGTGCACAAATCTTAAGTGCACAGCTGGGTAAACTTTTAC
AGTGTTCACCTGTGTAACTACCACCCGGATCAAGTTAGAGAACACTTCCATTGCCACAGAAGGCTTCCTA
TAGGTGTCTGTTCCCAGTTGATACCCATGACCCTCACCACCTCCAGAGGTCCCCACTGTTTTCACCCCAT
CGTCGCAGATTATTTTTTAATTTTATAATGTAGTATCTTTGTTCCTATGTATAGCAGGAGTTCATTTTCA
TTGCTCTTGCATTTGTATGAATATACTAGAATTTATTCATCCATTCTAATGTTAATGGACATTTGAATTA
TTTCCAGTTTGGGGCTGTTAAGCATAATGCTGCTAAGAACATTCTTATCTTTTCTGTTGGGTATATATAC
AGGAATGGAATTGCTGAGTCACGGGGTATATTTCTGTTTAGCTTTAGTAAATTCTGCCAGTTTACACTCC
TACCAGCAATGTATGAGAGTTTTAGTTGTTCACCATTCATAACACTGTTAGCAGCCCTTTTCGTTTTAGA
CTGGTTGGCTGTTCACAAATTTTGATATTCCTTAAAGCATTAAAACATTTTAATAAATACTTATAAATAG
GTA

The enzyme, SPRTN, is a DNA-dependent metalloprotease. It is capable of proteolytic digestion of the protein component of TOP1ccs (i.e. TOP1—see Examples below) and cleaving DNA-binding proteins in their unstructured region, such as by histones H2A, H2B, H3, H4 and DNA-binding proteins that should be firstly unfolded such as Topoisomerase 1 and 2. SPRTN enzyme activity may, therefore, be detected in vitro or ex vivo by performing a cleavage assay using the TOP1 protein as a substrate. The amino acid sequence of one embodiment of human SPRTN is referred to here:

sprT-like domain-containing protein Spartan isoform a [Homo sapiens]
NCBI Reference Sequence: NP_114407.3
[SEQ ID NO: 4]
MDDDLMLALRLQEEWNLQEAERDHAQESLSLVDASWELVDPTPDLQALFVQFNDQFFWGQLEAVEVKWSV
RMTLCAGICSYEGKGGMCSIRLSEPLLKLRPRKDLVETLLHEMIHAYLFVTNNDKDREGHGPEFCKHMHR
INSLTGANITVYHTFHDEVDEYRRHWWRCNGPCQHRPPYYGYVKRATNREPSAHDYWWAEHQKTCGGTYI
KIKEPENYSKKGKGKAKLGKEPVLAAENKDKPNRGEAQLVIPFSGKGYVLGETSNLPSPGKLITSHAINK
TQDLLNQNHSANAVRPNSKIKVKFEQNGSSKNSHLVSPAVSNSHQNVLSNYFPRVSFANQKAFRGVNGSP
RISVTVGNIPKNSVSSSSQRRVSSSKISLRNSSKVTESASVMPSQDVSGSEDTFPNKRPRLEDKTVFDNF
FIKKEQIKSSGNDPKYSTTTAQNSSSSSSQSKMVNCPVCQNEVLESQINEHLDWCLEGDSIKVKSEESL

The SPRTN enzyme may be observed in three isoforms, isoform a (489 amino acids; Accession: NP_114407.3 GI: 58331105), isoform b (250 aa protein Accession: NP_001010984.1 GI: 58331107) and isoform c (207 aa protein; Accession: NP_001248391.1 GI: 387762597)

In another aspect of the invention, there is provided a method of treating a cancer in a subject in need thereof, the method comprising:
i. identifying a subject predicted to respond to therapy with a TOP1 inhibitor according to a method of the invention; and
ii. administering a TOP1 inhibitor to the identified subject.

In another aspect of the invention, there is provided a method of treating a cancer in a subject, the method comprising administering a TOP1 inhibitor to a subject wherein the level of SPRTN and/or SPRTN mRNA in a sample of primary colorectal cancer cells from the subject is low. Preferably the cancer to be treated is a metastatic colorectal cancer.

In a yet further aspect of the invention, there is provided a TOP1 inhibitor for use in treating a cancer in a subject, wherein the subject has a low level of SPRTN enzyme and/or SPRTN mRNA in a sample of cells of the cancer to be treated. Preferably the sample of cells is a sample of primary colorectal cancer cells and the cancer to be treated is a metastatic colorectal cancer.

According to another aspect of the invention, there is provided a method of selecting a cancer patient for treatment with a TOP1 inhibitor, the method comprising:
i. obtaining a sample of cancer cells from a cancer patient; and
ii. detecting SPRTN enzyme and/or SPRTN mRNA levels in the sample of cancer cells; and
iii. selecting the patient for treatment with a TOP1 inhibitor if the level of SPRTN enzyme and/or SPRTN mRNA in the sample is low.
   Preferably the sample of cells is a sample of primary colorectal cancer cells and the cancer to be treated is a metastatic colorectal cancer.

In a yet further aspect of the invention, there is provided a method of predicting if a cancer will respond to treatment with a TOP1 inhibitor, the method comprising:
i. obtaining a sample of cancer cells from a cancer patient; and
ii. detecting SPRTN enzyme and/or SPRTN mRNA expression in the sample of cancer cells; and
iii. predicting that if the level of SPRTN enzyme and/or SPRTN mRNA in the sample is low then the patient will respond to a TOP1 inhibitor.
   Preferably the sample of cells is a sample of primary colorectal cancer cells and the cancer to be treated is a metastatic colorectal cancer.

“Predicting if a cancer will respond to treatment with a TOP1inhibitor” may refer to recording the name or an identifier of the patient so that a third party is aware that the patient has a cancer that is expected to respond to treatment with a TOP1 inhibitor, or has a cancer that is not expected to respond to treatment with a TOP1 inhibitor.

The term “selecting” can refer to recording the name or an identifier of the patient so that a third party is aware that the patient is intended to receive treatment with a cancer therapy.

The term “recording” can refer to writing, typing, digitally noting or fixation.

The subject or patient may be a mammal and is preferably a human, but may alternatively be a monkey, ape, cat, dog, cow, horse, rabbit or rodent.

There may be no detectable or low levels of SPRTN enzyme and/or SPRTN mRNA in a sample of cancer cells, in which case the cancer or metastatic cancer derived therefrom may be predicted to respond to treatment with a TOP1 inhibitor or the patient may be selected for treatment with a cancer therapy. There may be high levels of SPRTN enzyme and/or SPRTN mRNA in a sample of cancer cells, in which case the cancer or metastatic cancer derived therefrom may be predicted to not respond to treatment with a TOP1 inhibitor or the patient may not be selected for treatment with a cancer therapy. Preferably the sample of cells is a sample of primary colorectal cancer cells and the cancer to be treated is a metastatic colorectal cancer.

The term “respond to treatment or therapy” may refer to killing cancer cells, in particular metastatic cancer cells, and/or shrinking a tumour, in particular a metastatic tumour, stopping or reducing replication of cancer cells or stopping or reducing growth of a tumour and/or its metastasis.

The method provides a robust biomarker for identifying a cancer patient, in particular a patient with metastatic colorectal cancer, that is sensitive to treatment with a TOP1 inhibitor.

The skilled person would know how to detect the SPRTN enzyme protein. For example, detecting SPRTN enzyme in the sample of primary cancer cells may comprise the use of any one of the following techniques: chromogenic (enzyme activity) assays and/or fluorometric imaging plate reader (FLIPR) assays; flow cytometry; immunoassays, such as enzyme-linked immunosorbent assays (ELISAs), an enzyme immunoassay (EIAs), radioimmunoassay (RIAs), Western Blots, immuno-precipitation or immunohistochemistry; immunofluorescence; chromogenic (enzyme activity) assays; fluorometric imaging plate reader (FLIPR) assay; high performance liquid chromatography (HPLC) tandem mass spectrometry (MS/MS); and a biochip. Preferably, SPRTN enzyme is detected by immunohistochemistry, immunofluorescence or ELISA. Preferably, SPRTN enzyme expression is detected by immunohistochemistry or immunofluorescence.

The skilled person would know how to detect the SPRTN mRNA. For example, detecting SPRTN mRNA in the sample of primary cancer cells may comprise the use of any one of the following techniques RNA sequencing, in situ hybridisation, RNA microarrays, RT-PCR, qPCR or any quantifiable method of scoring RNA expression.

The level of SPRTN enzyme and/or SPRTN mRNA in cells of a sample may be compared to the level of SPRTN enzyme and/or SPRTN mRNA in cells of a reference sample.

The reference sample may be a sample of cancerous cells. The reference sample may be a sample of non-cancerous cells. Preferably, the reference sample is from the same tissue as the cancer sample. The reference sample may be from the subject with cancer. Alternatively, the reference sample may be from a different subject, preferably, the same tissue as the cancer sample.

The sample may be a sample of the cancer cells to be treated, such as tumour tissue. The sample may be a biopsy. The skilled person would appreciate, for example, that if the cancer to be treated originates in the bowel (i.e. is bowel cancer), the sample may include a sample or biopsy of the bowel. However, the cancer may metastasize, in which case the sample may be taken from sites that the cancer has metastasized to. Thus, the sample may comprise tissue, blood, plasma, serum or spinal fluid. The sample may comprise cancer cells from a primary cancer, and the cancer to be treated may be a metastatic cancer derived from the primary cancer.

The method of the invention may be carried out in vitro or ex vivo. The cells being tested may be in a tissue sample (for ex vivo based tests) or the cells may be grown in culture (an in vitro sample).

The step of obtaining the sample cells may not form part of the method of the invention.

The cancer therapy may be a TOP1 inhibitor. A TOP1 inhibitor may be an agent that inhibits the enzyme activity of TOP1 by binding to the active site of the enzyme or by binding allosterically. A TOP1 inhibitor may be selected from the group comprising or consisting of camptothecin, an irinotecan, topotecan, lamellarin D, rubitecan, exatecan, bleotecan, 7-ethyl-10-hydroxycamptothecin (SN 38), all derivatives based on camptothecin, and other DNA Topoisomerase 1 inhibitors.

The cancer may be leukaemia, for example acute myeloid leukaemia (AML), chronic myeloid leukaemia (CML), acute lymphocytic leukaemia (ALL), chronic lymphocytic leukaemia (CLL), T-cell prolymphocytic leukaemia (T-PLL) and/or hairy cell leukaemia. In one embodiment, the cancer is chronic lymphocytic leukaemia (CLL).

The cancer may be selected from the group comprising or consisting of anal cancer, bladder cancer, bone cancer, brain cancer, breast cancer, cervical cancer, colorectal cancer, colon cancer, endometrial cancer, head and neck cancer, leukaemia, liver cancer, lung cancer, non-small cell lung carcinoma, kidney cancer, mouth cancer, ovarian cancer, pancreatic cancer, prostate cancer, skin cancer, rectal cancer, stomach cancer, testicular cancer, throat cancer, thyroid cancer, urethral cancer, vulvar cancer, and any combination thereof. The cancer may be colon cancer, colorectal cancer, pancreatic cancer, ovarian cancer and/or non-small cell lung carcinoma. In one embodiment, the cancer is colon cancer or colorectal cancer. Preferably, the cancer is colorectal cancer and/or metastatic colorectal cancer.

The skilled person will understand that optional features of one embodiment or aspect of the invention may be applicable, where appropriate, to other embodiments or aspects of the invention.

Embodiments of the invention will now be described in more detail, by way of example only, with reference to the accompanying figures.

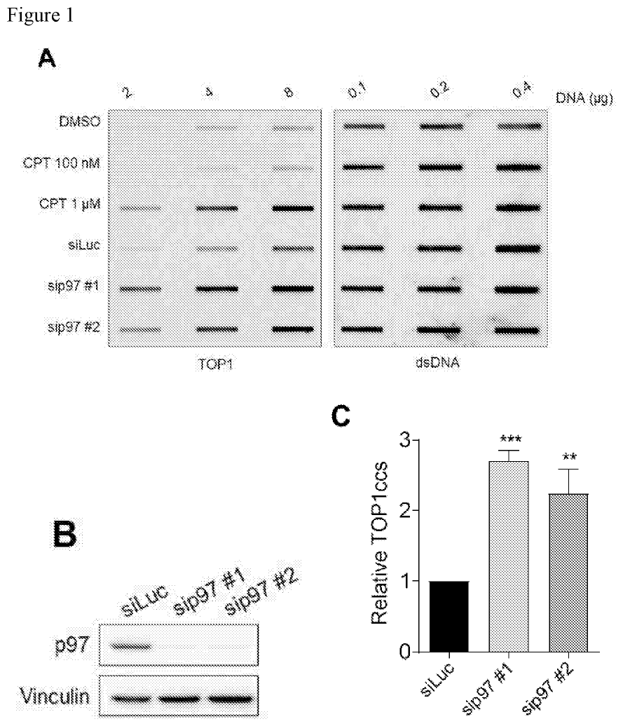

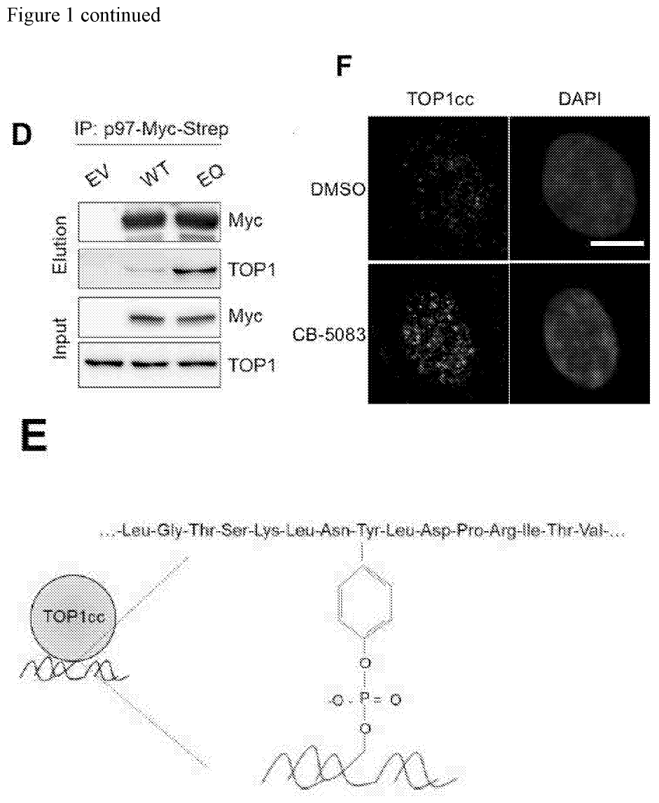

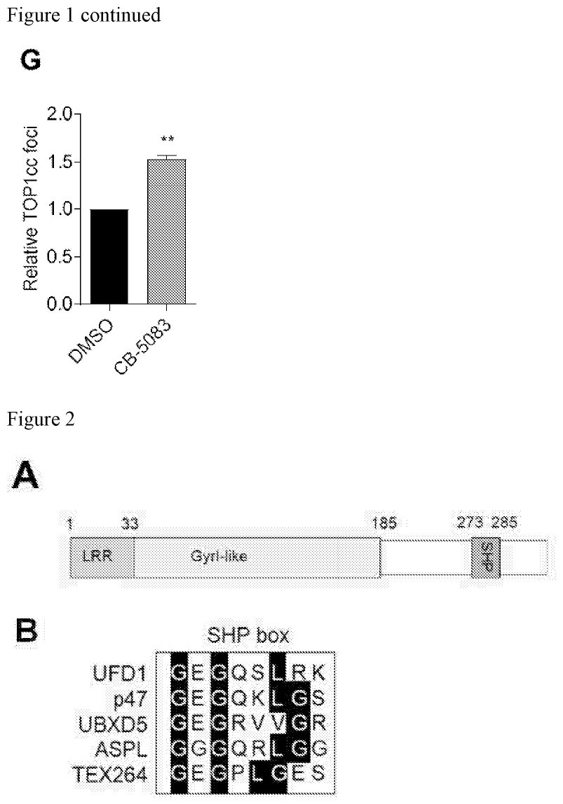

FIG. 1 shows that the ATPase p97 is involved in TOP1cc repair. FIG. 1A, RADAR assay to assess TOP1cc accumulation after short interfering (si)RNA-mediated depletion of p97. Treatment with 1 μM CPT for 1 hour was used as a positive control for TOP1cc induction. Double-stranded (ds)DNA is used as a loading control. FIG. 1B, Immunoblot to confirm p97 depletion. FIG. 1C, Quantification of Figure 1A (error bars represent mean ±SEM; n=3; ***P<0.001, **P<0.01; Student's t-test). FIG. 1D, Immunoblots of anti-Strep-tag immunoprecipitates prepared from HEK293 transiently expressing wild-type (WT) or ATPase-deficient (E578Q/EQ) p97-Myc-Strep. EV denotes empty vector. FIG. 1E, Schematic diagram of the antigen recognised by the TOP1cc-specific antibody. FIG. 1F, Representative nuclei of RPE-1 cells, treated with either DMSO or the p97 inhibitor CB-5083 for 4 hours, then immunostained with the TOP1cc-specific antibody. DAPI, 4′,6-diamidino-2-phenylindole. Scale bar is 10 μm. FIG. 1G, Quantification of Figure 1F (error bars represent mean ±SD; n=2; **P<0.01; Student's t-test).

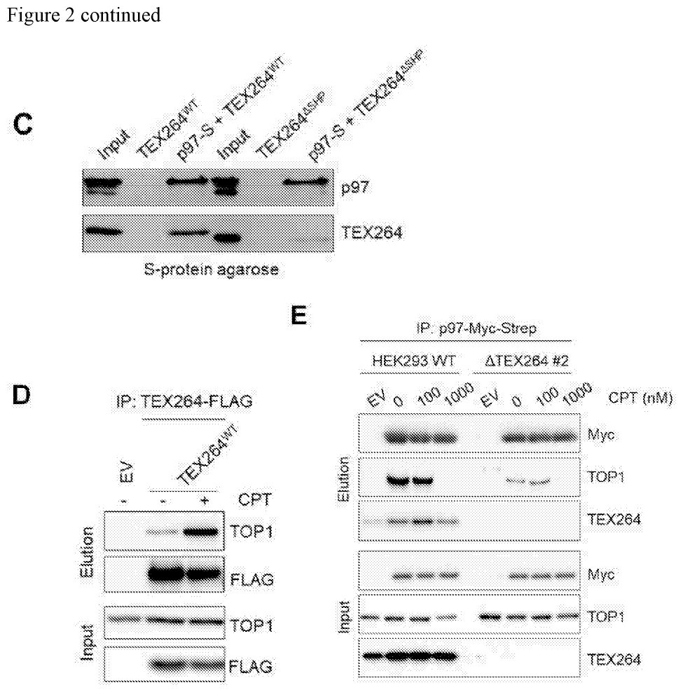

FIG. 2 shows that TEX264 Recruits p97 to TOP1. FIG. 2A, Schematic diagram of the TEX264 protein. LRR denotes leucine-rich repeat; Gyrl-like, Gyrase inhibitory-like; SHP, SHP box. FIG. 2B, Alignment of the SHP box of known p97 cofactors with that of TEX264. Conserved residues are highlighted in black. FIG. 2C, In vitro p97 pulldown experiments after incubation of recombinant p97-S tag with His-tagged TEX264WT or 554 TEX264ΔSHP. FIG. 2D, FLAG immunoprecipitates prepared from HEK293 cells transiently expressing TEX264WT-FLAG cDNA or EV, treated with CPT (25 nM) or DMSO for 1 hour. FIG. 2E, Immunoblots of anti-Strep-tag immunoprecipitates prepared from wild-type (WT) or ΔTEX264 HEK293 cells expressing p97-Strep-Myc.

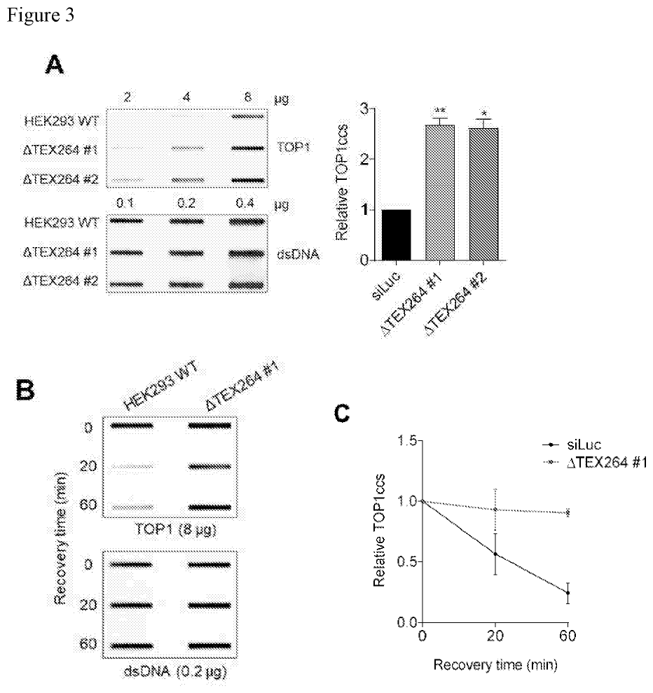

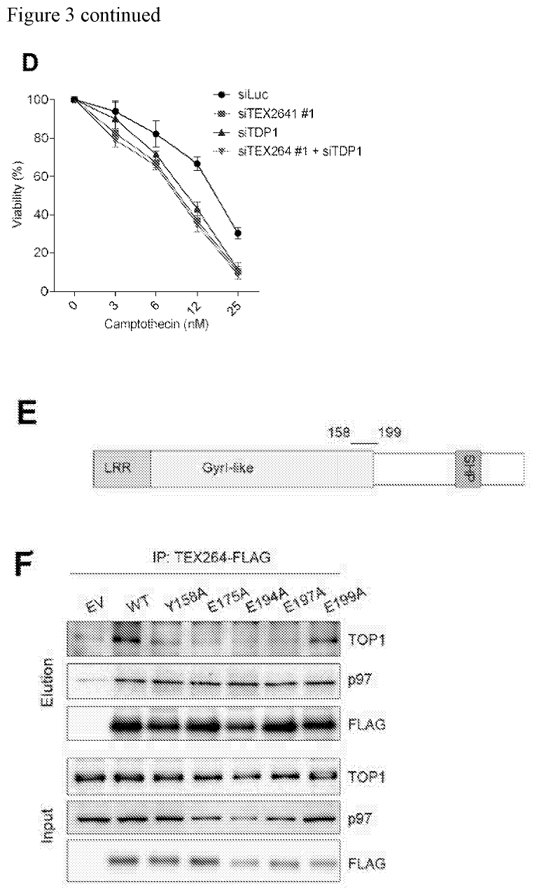

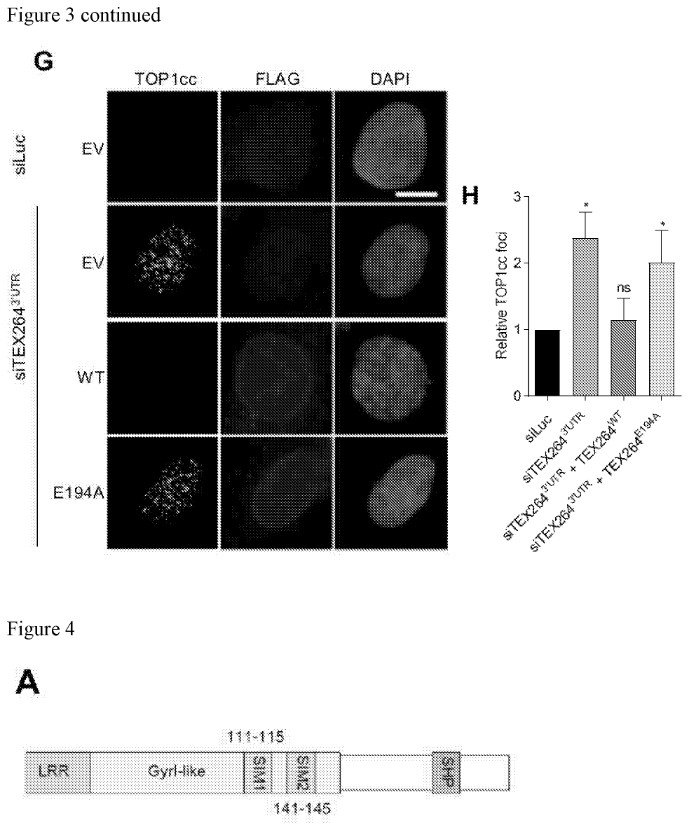

FIG. 3 shows that TEX264 counteracts TOP1cc accumulation. FIG. 3A, Slot blot analysis of TOP1ccs prepared from WT or ΔTEX264 HEK293 cells using the RADAR assay. DPCs were probed with a TOP1 antibody. Corresponding quantifications on the right (error bars represent mean ±SEM; n=3; **P<0.01, *P<0.05; Student's t-test) FIG. 3B, TOP1ccs isolated by RADAR from WT or ΔTEX264 HEK293 cells treated for 1 hour with CPT (50 nM), then released into CPT-free media for 20 or 60 minutes. FIG. 3C, Quantification of FIG. 3B (error bars represent mean±SEM; n=3). FIG. 3D, Colony forming assay to assess the viability of HeLa cells transfected with the indicated siRNAs. Cells were treated for 24 hours with the indicated doses of CPT, released into normal media for 7 days, then fixed and stained. Viability represents the number of colonies in each sample expressed as a percentage of the number of colonies formed in the corresponding untreated sample (error bars represent mean±SEM; n=3). FIG. 3E, Schematic of the TEX264 protein, indicating the location of residues mutated in FIG. 3F. FIG. 3F, FLAG immunoprecipitates prepared from ΔTEX264 HEK293 cells transiently expressing the indicated versions of FLAG-tagged TEX264. FIG. 3G, Representative images of U2OS cells treated with siLuc or siTEX2643′UTR, transfected with the indicated FLAG-tagged variants of TEX264, and immunostained with TOP1cc (green) and FLAG (red) antibodies. Scale bar, 10 μm. FIG. 3H, Quantification of FIG. 3G (error bars represent mean ±SEM; n=3; *P<0.05; ns, not significant; Student's t-test).

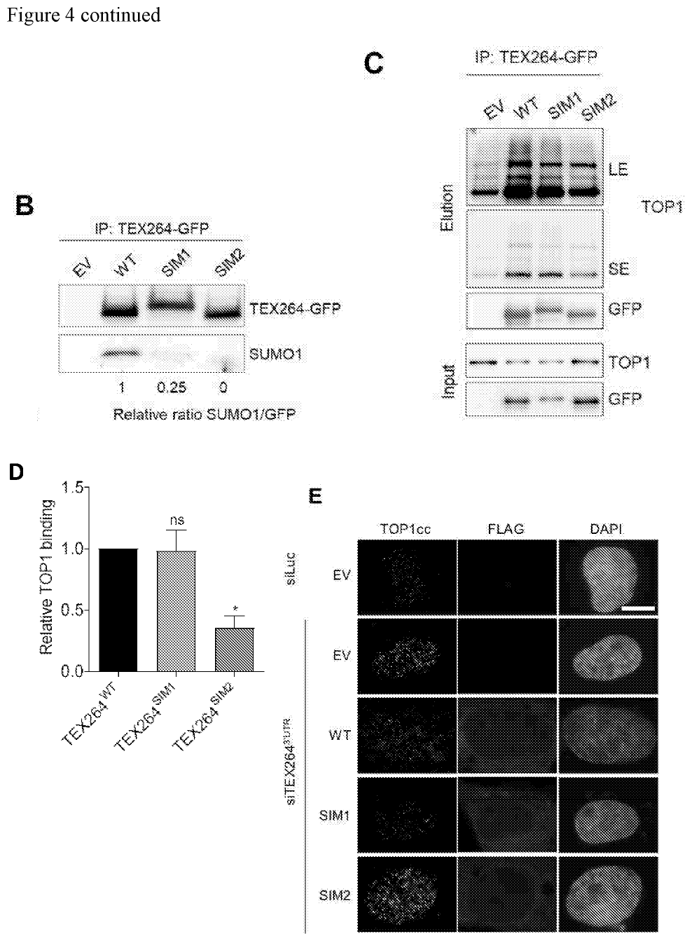

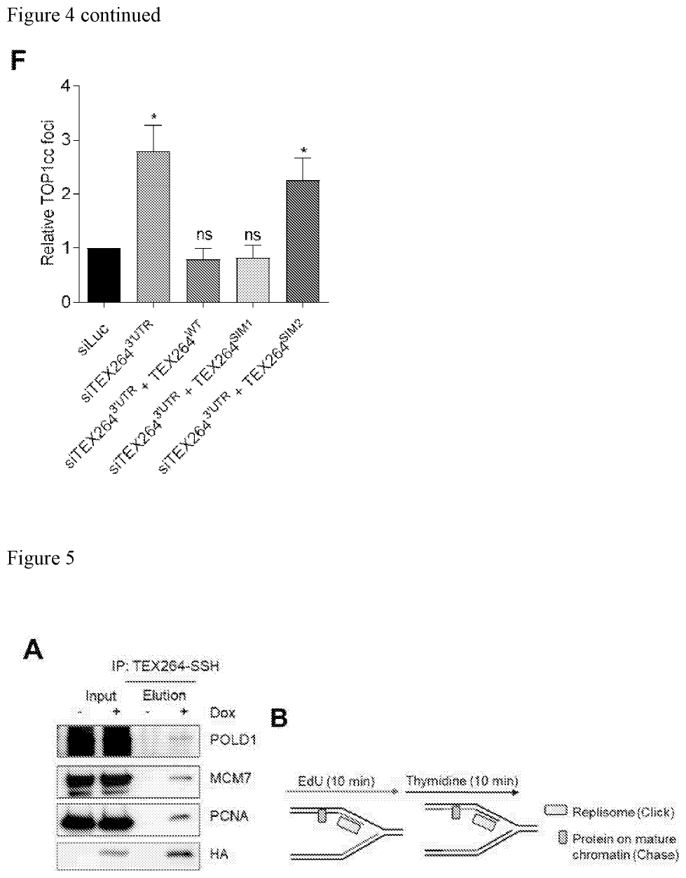

FIG. 4 shows that TEX264 recognises to SUMOylated TOP1. FIG. 4A, Schematic diagram of TEX264, indicating the location of its putative SUMO-interacting motifs (SIMs). FIG. 4B, Immunoblot of GFP & SUMO1 after incubation of purified GFP-tagged WT & SIM mutant TEX264 with free SUMO1. FIG. 4C, Immunoblot analysis of GFP immunoprecipitates prepared from ΔTEX264 HEK293 cells transiently expressing the indicated GFP-tagged versions of TEX264. LE & SE denote long & short exposure, respectively. FIG. 4D, Quantification of C (error bars represent mean ±SD; n=2; *P<0.05; ns, not significant; Student's t-test). FIG. 4E, Immunofluorescent detection of TOP1ccs (green) and TEX264-FLAG (red) in U2OS cells transfected with the indicated siRNAs and cDNAs. Scale bar, 10 μm. FIG. 4F, Quantification of experiments represented in E (error bars represent mean ±SEM; n=3; *P<0.05; ns, not significant; Student's t-test).

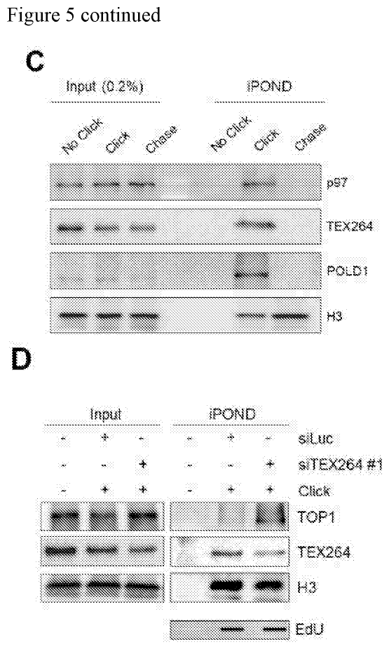

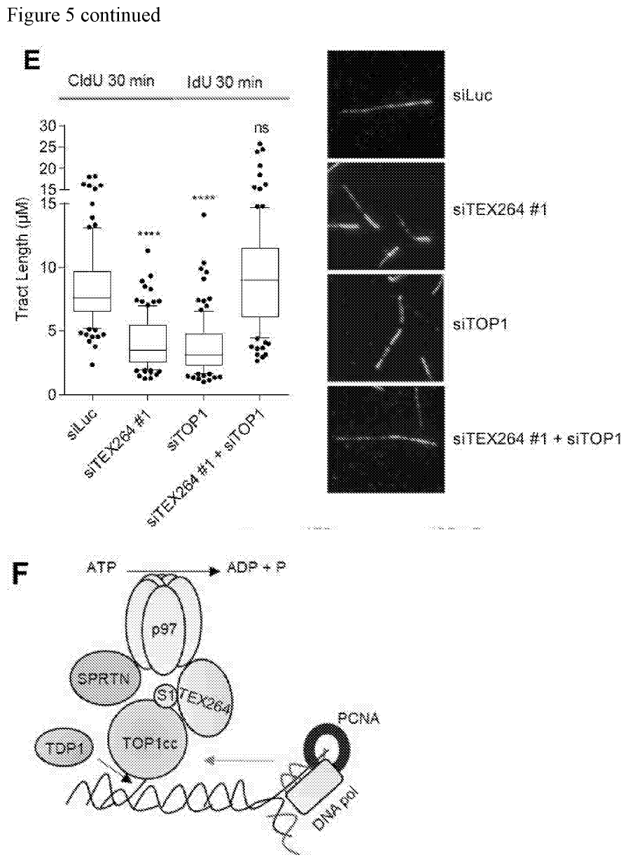

FIG. 5 shows that TEX264 Acts at replisomes. FIG. 5A, Immunoblots of anti-HA immunoprecipitates prepared from doxycycline (Dox)-inducible TEX264-SSH HEK293 Flp-In TRex cells, treated with and without Dox. FIG. 5B, Schematic of iPOND approach. FIG. 5C, iPOND in HEK293 cells showing the presence of p97 and TEX264 at replication forks (click) and their absence on the chromatin behind replication forks (chase). FIG. 5D, iPOND analysis of TOP1 at replication forks in HEK293 cells transfected with the indicated siRNA. A slot blot analysis of biotin-conjugated EdU using a Streptavidin-HRP antibody was performed to ensure equal amounts of EdU-labelled DNA were isolated from each sample. FIG. 5E, (Above) Schematic of DNA fibre assay. (Below) DNA fibre analysis of HEK293 cells treated with the indicated siRNAs and labelled with CldU (30 min; red), followed by IdU (30 min; green). Quantification of IdU track lengths is shown. At least 100 fibres were measured per condition. Whisker box plots show mean values and data within the 10-90 percentile. ****P<0.0001; ns, not significant; two-tailed Mann-Whitney test. Representative DNA fibres are shown on the right. FIG. 5F, Model: TEX264 recruits p97-SPRTN sub-complexes to SUMOylated TOP1ccs to facilitate their processing upstream of TDP1. S1, denotes SUMO1; DNA pol, DNA polymerase.

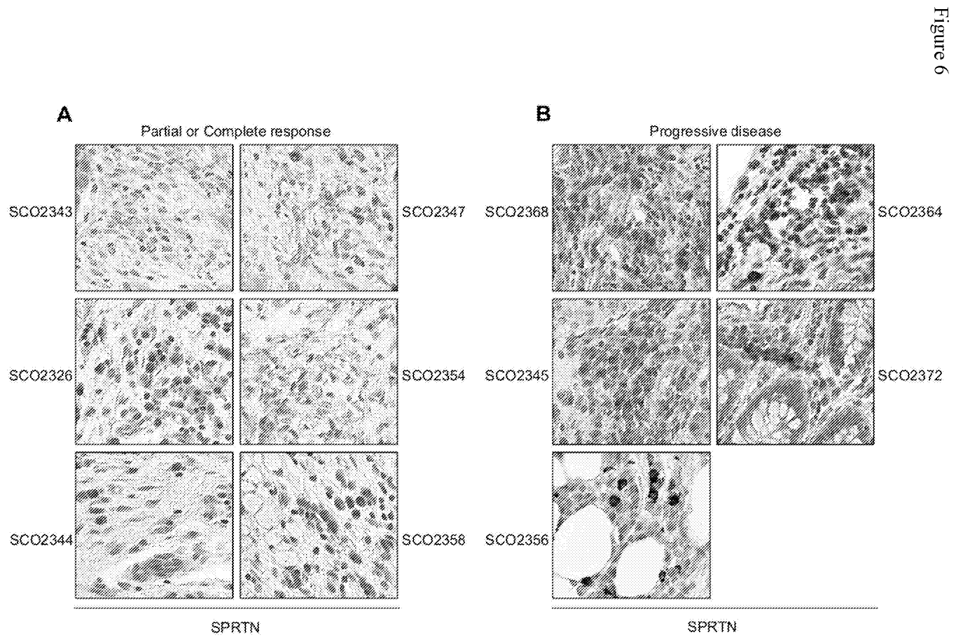

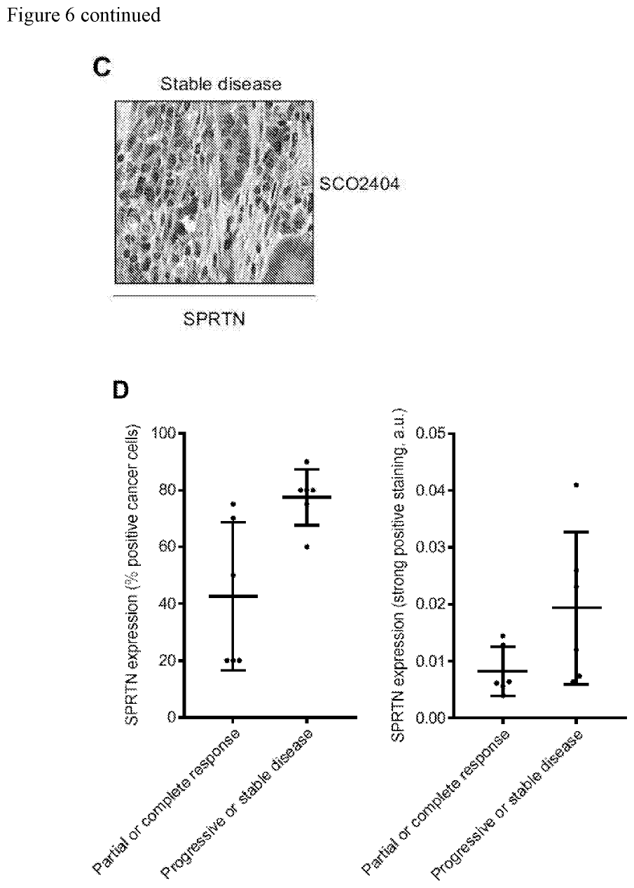

FIG. 6 shows that SPRTN expression correlates with irinotecan resistance in metastatic colorectal cancer. Histological analysis of SPRTN expression in primary human colorectal tumours before FOLFIRI treatment. FIG. 6A, SPRTN expression in patients who exhibited partial or complete response to therapy. FIG. 6B, SPRTN expression in patients with progressive disease. FIG. 6C, SPRTN expression in a patient with table disease after therapy. FIG. 6D, Quantification of SPRTN-positive cancer cells (left) and ‘strong’ positive SPRTN staining (right). a.u. denotes arbitrary units.

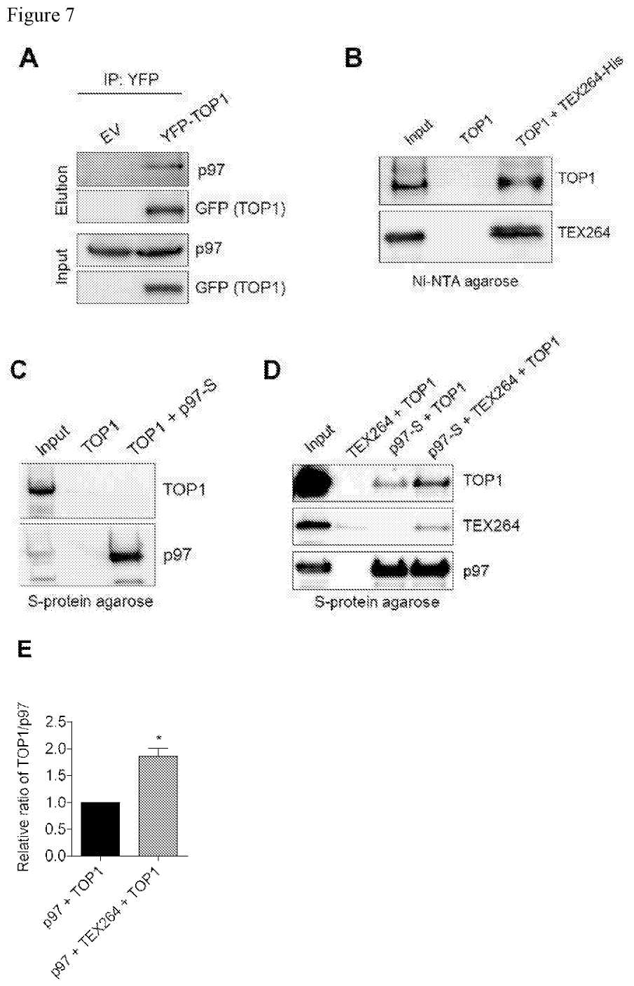

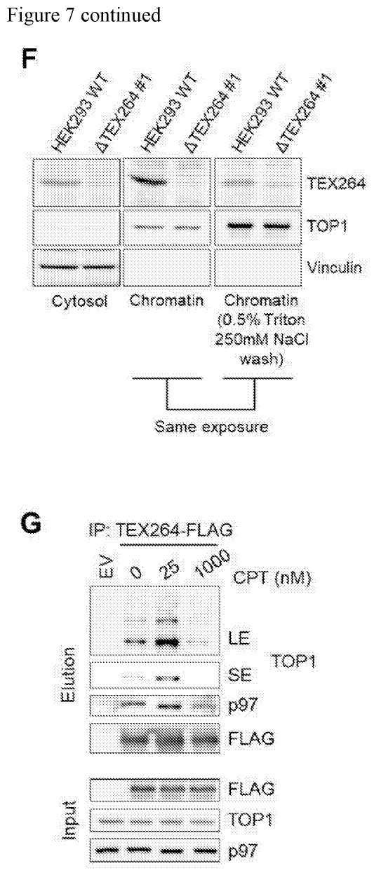

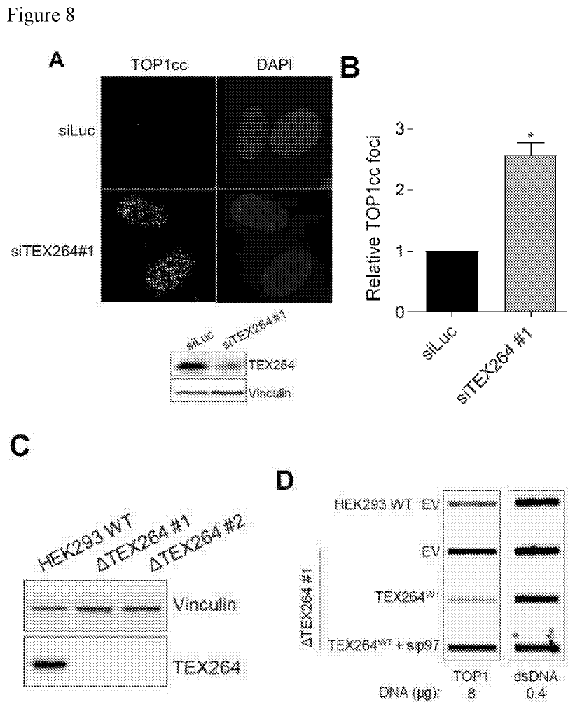

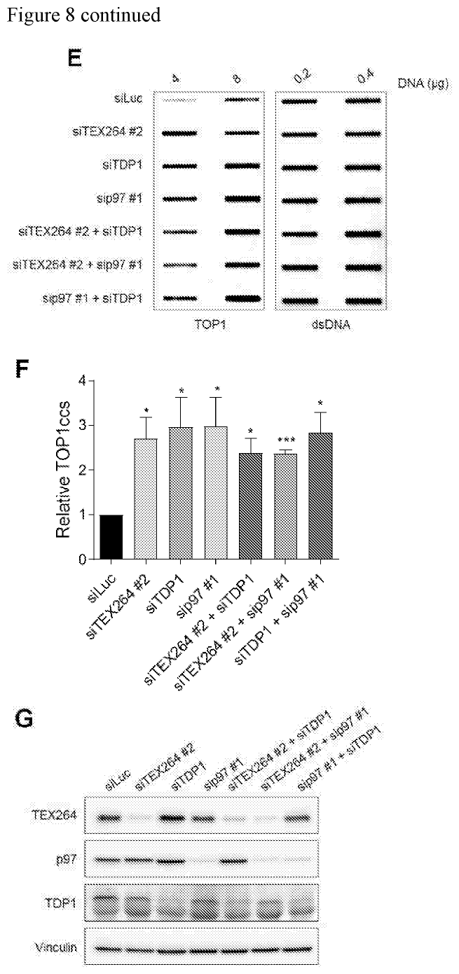

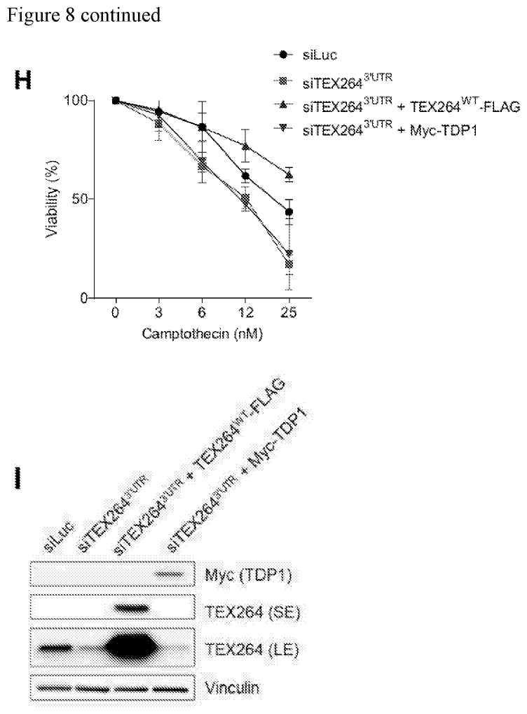

FIG. 7 shows that TEX264 recruits p97 to TOP1. FIG. 7A, Co-immunoprecipitation of p97 with chromatin-bound YFP-TOP1. FIG. 7B, In vitro interaction (pull down) assay using recombinant TOP1 incubated with or without His-tagged TEX264. FIG. 7C, In vitro interaction assay using recombinant TOP1 incubated with or without S-tagged p97. FIG. 7D, Pull-down of recombinant p97-S after incubation with TOP1, with or without TEX264. FIG. 7E, Quantification of FIG. 8D (error bars represent mean ±SD; n=2; *P<0.05; Student's t-test). FIG. 7F, Cytosolic and chromatin fractions of WT and TEX264-knockout (ΔTEX264) HEK293 cells immunoblotted with the indicated antibodies. Chromatin was washed with 0.5% Triton X-100 and 250 mM NaCl to separate loosely-bound and tightly-bound protein fractions. FIG. 7G, Immunoblot analysis of TEX264-FLAG immunoprecipitates prepared from HEK293 cells treated with the indicated doses of CPT for 1 hour.

FIG. 8 shows that TEX264 counteracts TOP1cc accumulation. FIG. 8A, Immunofluorescent detection of TOP1ccs (green) in RPE-1 cells transfected with the indicated siRNAs. (Below) immunoblot to confirm depletion. FIG. 8B, Quantification of FIG. 8A (error bars represent mean ±SD; n=2; *P<0.05; Student's t-test). FIG. 8C, Immunoblot analysis of TEX264 expression in WT and ΔTEX264 HEK293 cells. FIG. 8D, RADAR analysis of TOP1ccs in WT and ΔTEX264 HEK293 cells treated with the indicated siRNAs and transfected with EV or cDNA encoding TEX264WT-SSH. FIG. 8E, RADAR to assess TOP1ccs in HEK293 cells treated with the indicated siRNAs. FIG. 8F, Quantification of FIG. 8E (error bars represent mean ±SD; n=2; *P<0.05; ***P<0.001; Student's t-test). FIG. 8G, Immunoblot to confirm depletions in FIG. 8E. FIG. 8H, Colony forming assay to assess the survival of HeLa cells after CPT treatment. Cells were transfected with the indicated siRNA and cDNA. Cells were treated for 24 hours with the indicated doses of CPT, then allowed to recover for 7 days. Viability is expressed as a percentage of the corresponding untreated samples. FIG. 8I, Control immunoblots for FIG. 8H. LE & SE denote long & short exposure, respectively.

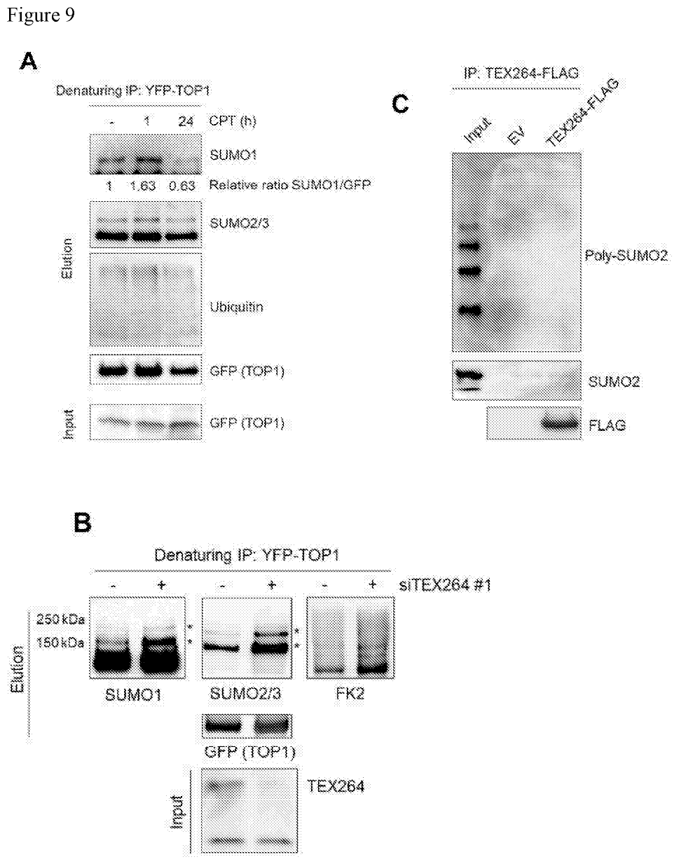

FIG. 9 shows that TEX264 is recruited to SUMOylated TOP1. FIG. 9A, Immunoblot analysis of SUMO1 & SUMO2/3 after denaturing immunoprecipitation of YFP-TOP1 from HEK293 cells treated or not with CPT (25 nM) for 1 or 24 hours. FIG. 9B, Immunoblot for SUMO1, SUMO2/3, and ubiquitin (FK2) after denaturing immunoprecipitation of YFP-TOP1 from HEK293 cells treated with the indicated siRNAs. Asterisks indicate SUMOylated TOP1. FIG. 9C, Immunoblot of FLAG & SUMO2/3 after incubation of free SUMO2 or poly-SUMO2 chains with purified FLAG-tagged TEX264.

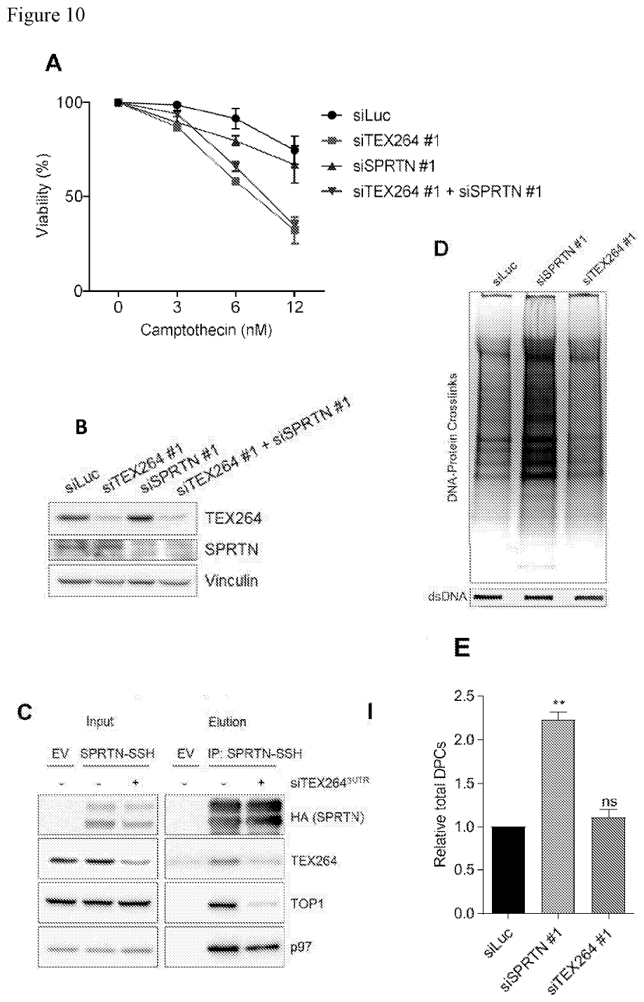

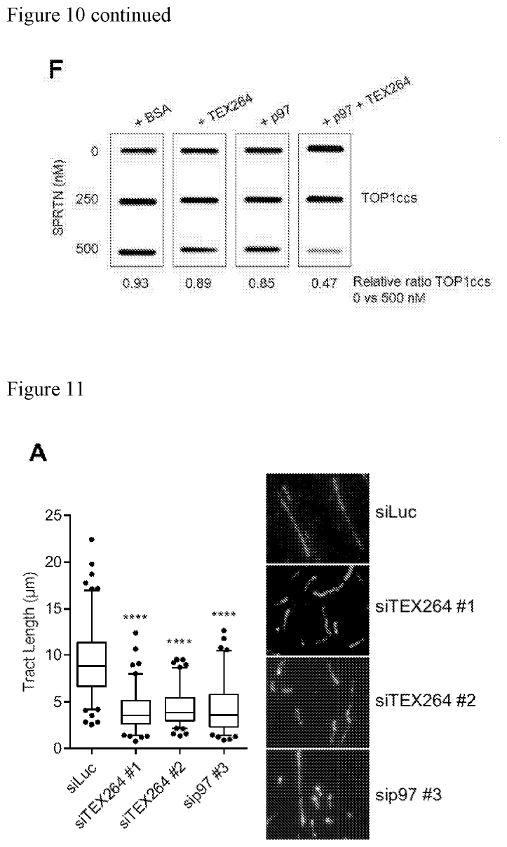

FIG. 10 shows that TEX264 Cooperates with the Metalloprotease SPRTN to resolve TOP1ccs. FIG. 10A, Colony forming assay to assess the survival of HeLa cells transfected with the indicated siRNAs following a 24 hour treatment with the indicated doses of CPT. FIG. 10B, Immunoblots to confirm depletion efficacy in A. FIG. 10C, Analysis of anti-Strep-tag immunoprecipitates prepared from HEK293 cells expressing SPRTN-SSH, transfected with the indicated siRNA. FIG. 10D, Total DNA-protein crosslinks (DPCs) isolated by RADAR from HEK293 cells treated with the indicated siRNAs. DPCs were resolved by SDS-PAGE and visualised by silver staining. FIG. 10E, Quantification of FIG. 10D (error bars represent mean ±SD; n=2; **P<0.01; ns, not significant; Student's t-test). FIG. 10F, In vitro TOP1cc repair assay. TOP1ccs were purified by CsCl-gradient centrifugation from HEK293 cells, incubated with the indicated purified proteins (100 nM/reaction), slot-blotted onto a nitrocellulose membrane and probed with a blotted -specific antibody.

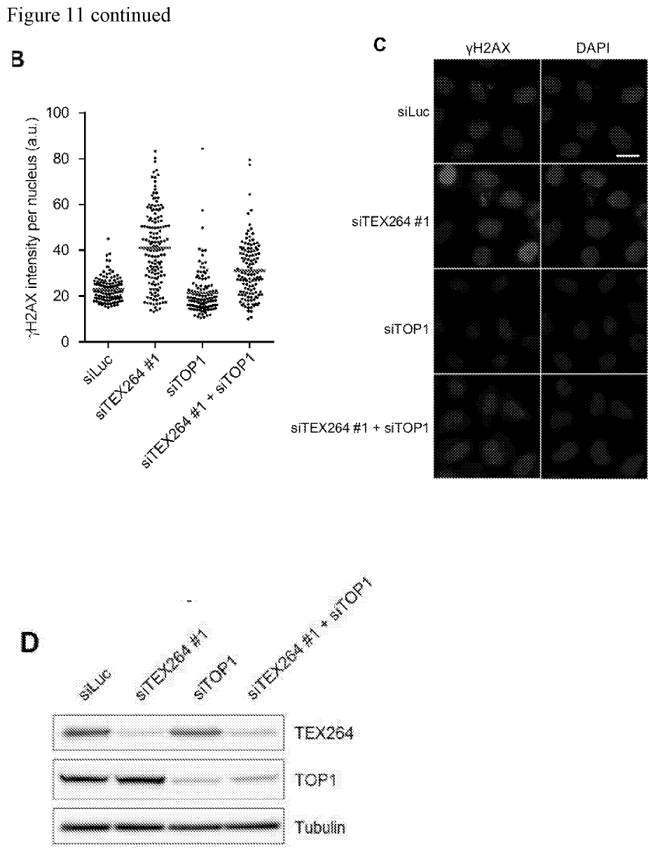

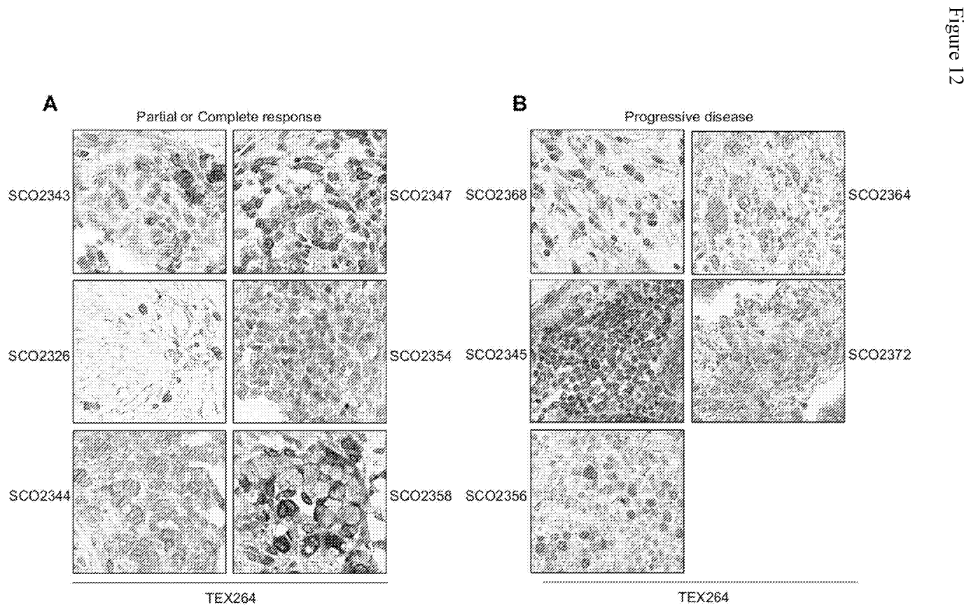

FIG. 11 shows that TEX264 acts at replisomes. FIG. 11A, DNA fibre analysis of replication fork velocity in HEK293 cells treated with the indicated siRNAs. IdU track lengths are shown. At least 100 fibres were measured per condition. ****P<0.0001; two-tailed Mann-Whitney test. Representative DNA fibres are shown on the right. FIG. 11B, Quantification of the mean nuclear γH2AX (phosphorylated on Ser139) intensity of HeLa cells treated with the indicated siRNAs. At least 100 nuclei were measured per condition and experiment. FIG. 11C, Representative images of nuclear γH2AX. Scale bar is 20 μm. FIG. 11D, Immunoblots to confirm the efficacy of TEX264 and TOP1 depletion.

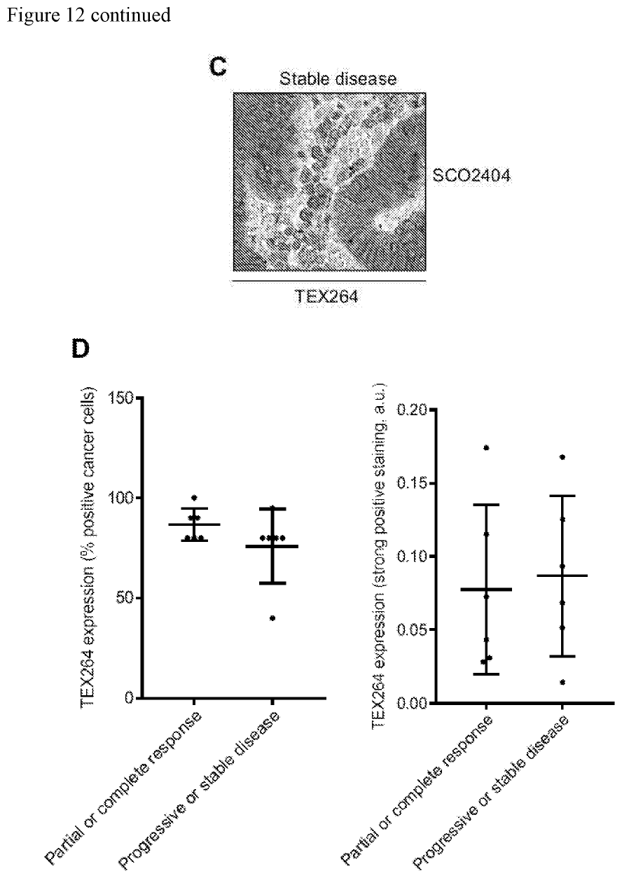

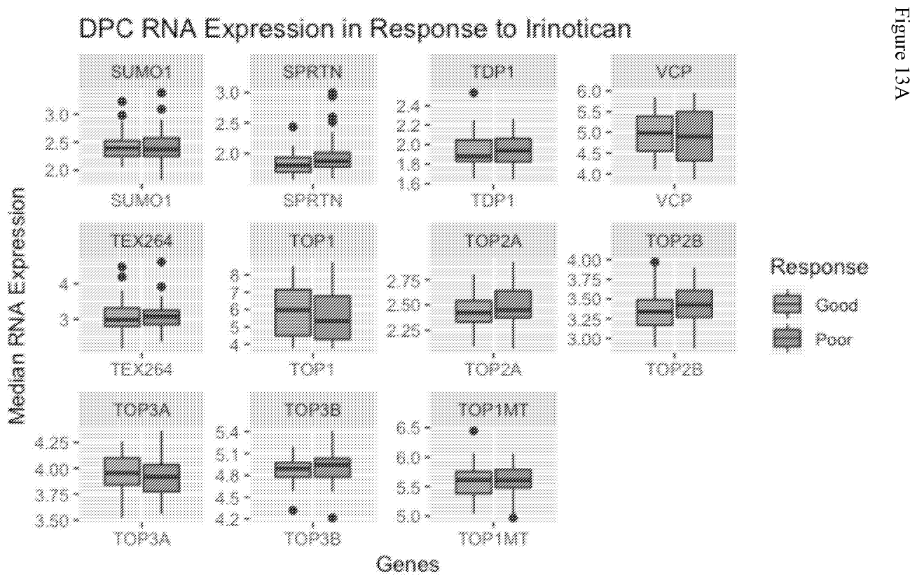

FIG. 12 shows TEX264 expression in metastatic colorectal cancers. Histological analysis of TEX264 expression in primary human colorectal tumours before FOLFIRI treatment. FIG. 12A, TEX264 expression in patients who exhibited partial or complete response to therapy. FIG. 12B, TEX264 expression in patients with progressive disease. FIG. 12C, TEX264 expression in a patient with stable disease after therapy. FIG. 12D, Quantification of TEX264-positive cancer cells (left) and ‘strong’ positive TEX264 staining (right).

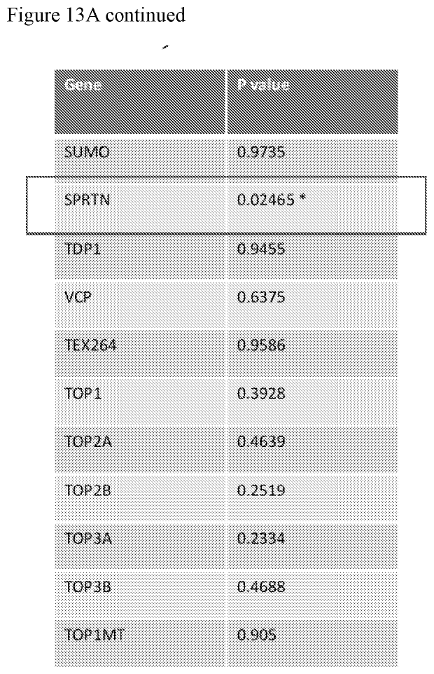

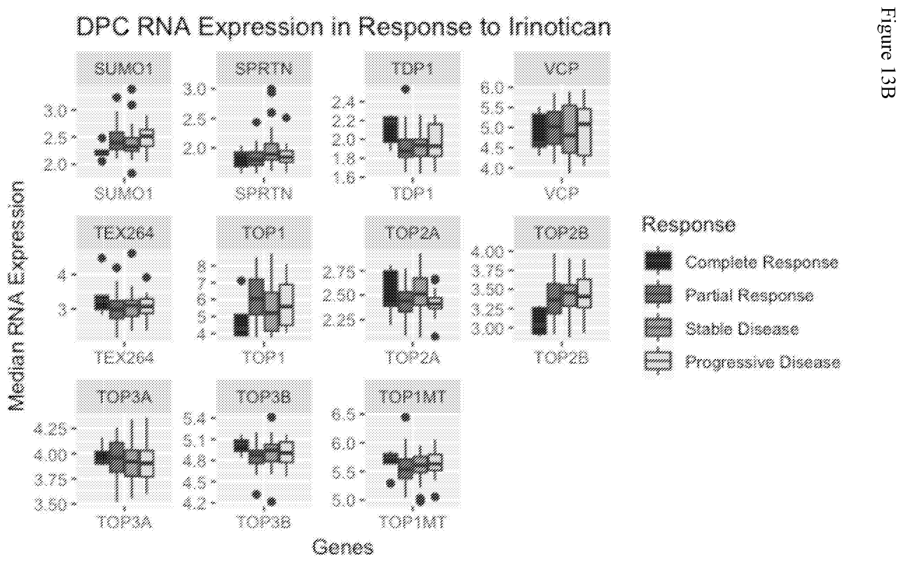

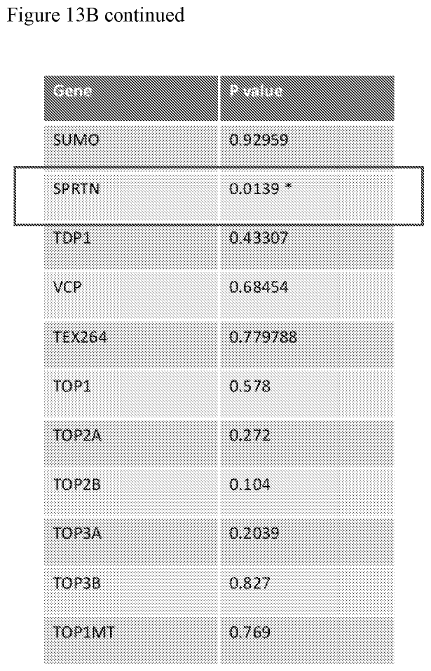

FIG. 13 shows mRNA expression of biopsies from colorectal cancer patients analysed by RNA sequencing. Known (TDP1, SUMO) or here identified genes (SPRTN, TEX264, VCP) involved in Top 1-ccs repair and Topoisomerases 1, 1MT, 2A, 2B, 3A, 3B were selected and statistically analysed for a correlation between their mRNA expression and metastatic colorectal cancer patients' response to irinotecan therapy. In total 78 patients have been analysed by two statistical tests: FIG. 13A T-test (comparing good patients' response vs bad patients' response) and FIG. 13B linear regression (comparing mRNA expression in complete, partial, stable and progressive disease). Complete and partial patients' response are considered as a good patients' response to irinotecan therapy. Stable and progressive disease in patients after irinotecan therapy are considered as a bad patients' response. SPRTN (highlighted in a red rectangle and with an asterisk) is only protein among analysed proteins, which low mRNA expression significantly corelates with a good patients' response to irinotecan therapy. SPRTN was the only protein which displayed high mRNA expression which significantly correlated with a poor patients' response to irinotecan therapy.

### EXAMPLES

The mechanisms of TOP1cc repair are not well defined. TDP1 can directly hydrolyse the bond that links TOP1 to DNA. However, it has long been known that TDP1 cannot act alone; its active site is too small to gain access to its substrate bond, which is protected within the TOP1cc structure. The inventors therefore decided to identify new components of the TOP1cc repair machinery.

A crucial role for the uncharacterised protein, TEX264, in TOP1cc repair has been identified (see Examples 2 to 6). TEX264 is a novel cofactor of the ATPase p97 (see Example 1) and is required to recruit p97 and SPRTN to TOP1ccs. TEX264 specifically recognises SUMO, making it the first known SUMO-specific p97 cofactor in metazoans (see Examples 2 and 4). It has been demonstrated that TOP1cc repair in human cells is distinct from yeast. Specifically, p97-TEX264-SPRTN and TDP1 are the components of the same pathway in human cells whereas in yeast Cdc48 and TDP1 act in two parallel pathways. This is especially important for our understanding of cancer therapy and resistance to topoisomerase poisons.

Finally, SPRTN expression strongly correlates with resistance to the TOP1 poison, irinotecan, in a cohort of patients with metastatic colorectal cancer (see Example 7). Thus, a unique and crucial role for TEX264 has been identified. It provides a mechanistic basis for the involvement of the p97-SPRTN complex in TOP1cc repair, and strongly suggests that this pathway is a clinically relevant target for chemotherapeutic intervention, particularly in colorectal cancer.

### MATERIALS AND METHODS

### Cell Culture

Human HEK293, U205, RPE-1, and HeLa cells were obtained from ATCC. All cell lines were cultured in DMEM containing 10% FBS and 5% Penicillin/Streptomycin. All cell lines were regularly screened for mycoplasma using a MycoAlert™ Mycoplasma Detection Kit.

### Generation of Cell Lines

To generate CRISPR-Cas9 TEX264 knockout cells, two plasmids —a CRISPR/Cas9 KO plasmid containing guide RNA targeting TEX264 (sc-417333), and a homology directed repair plasmid containing a puromycin resistance cassette (sc-417333-HDR)—were purchased from Santa Cruz. 2.5 μg of each plasmid was transfected into early-passage HEK293, HeLa, and U2OS cells using Fugene HD (Promega). After 72 hours, media supplemented with puromycin was added to the cells. The puromycin dose required to kill wild-type cells was determined to be: 1.25 μg/ml for HEK293 cells, 0.6 μg/ml for HeLa cells, and 1 μg/ml for U2OS cells. After 72 hours, the puromycin-containing media was removed and cells were sorted using a cell sorter into single-cell populations on a 96-well plate. TEX264 expression was analysed by immunoblotting. Multiple clones of each cell line showing loss of all detectable TEX264 were selected for subsequent analysis.

### Cellular Fractionations

Cells were resuspended in buffer A (10 mM Hepes pH 7.45, 10 mM KCl, 340 mM Sucrose, 10% Glycerol, Protease and Phosphatase Inhibitors, and 2 mM EDTA). Triton X-100 was added to a final concentration of 0.1% and cells were left on ice for five minutes. Cells were then centrifuged at 350×g for three minutes, the supernatant was collected as the cytosolic fraction, and the remaining pellet (nuclei) was then washed twice in buffer A without 0.1% Triton X-100. The nuclei were then resuspended in buffer B (3 mM EDTA, 0.2 mM EGTA, 5 mM Hepes pH 7.9, Protease and Phosphatase Inhibitors) and left on ice for 10 minutes. NP-40 was then added to a final concentration of 1% to remove membranes and was left to incubate on ice for a further five minutes. The nuclear fraction was then centrifuged at 1700×g for five minutes, the supernatant was collected as the nuclear soluble fraction. The remaining pellet was washed twice more in buffer B with 1% NP-40, and then twice in Benzonase buffer (50 mM Tris HC1 pH 7.9, 50 mM NaCl, 5 mM KC1, 3 mM MgCl₂, and protease and phosphatase inhibitors. The chromatin pellet was then centrifuged at 5000×g for five minutes and resuspended in Benzonase buffer, supplemented with 125 U of Benzonase (Merk Millipore) and incubated overnight rolling at 15 rpm at 4° C. The next morning, the Benzonase digestion was centrifuged at 20,000×g for five minutes, the supernatant was collected as the chromatin soluble fraction.

### Immunoprecipitation

Cells were lysed in IP lysis buffer (50 mM Tris, pH 7.4, 150 mM NaCl, 1 mM EDTA, 0.5% Triton X-100) or IP denaturing buffer (i.e. IP lysis buffer containing 1% SDS) supplemented with protease and phosphatase inhibitors and incubated on ice for 10 min. Denaturing IPs were quenched with Triton X-100 (to a final concentration of 1%). Chromatin was pelleted by centrifugation for 5 min at 1,000×g and then digested with Benzonase at room temperature in Benzonase buffer (2 mM MgCl₂, 50 mM Tris, pH 7.4, 150 mM NaCl). Lysates were incubated with anti-FLAG M2 agarose (Sigma-Aldrich), Strep-Tactin Sepharose (IBA), or GFP-trap agarose (Chromotek) for 1-2 h on a rotator at 4° C., washed three times with IP wash buffer (150 mM NaCl, 50 mM Tris-HC1; 0.5 mM EDTA), and resuspended in 2× Laemmli buffer.

### Protein Purification & In Vitro Interaction Assays

p97 was cloned into a pET21a vector and purified with a C-terminal S-Tag from Rosetta E. coli using standard methods. TEX264 was purified without its hydrophobic N-terminus (amino acids 1-25; ΔNT) to render the protein more soluble and enable purification. ΔNT TEX264 was cloned into a pET21a vector (with a C-terminal His-tag) and expressed in Rosetta E. coli. Cell pellets were resuspended in denaturing resuspension buffer (800 mM NaCl, 20 mM Tris HC1 pH 7.0, 8 M Urea) containing 1% Triton X-100 and PMSF, before adding lysozyme and incubating at room temperature for 30 minutes. Cell lysates were then incubated with Ni-NTA agarose beads (Qiagen) for two hours at room temperature. Proteins were refolded by washing three times in 1× denaturing resuspension buffer (without Triton X-100), containing decreasing concentrations of Urea (6 M, 4 M, 2 M, 0 M). Proteins were eluted from the beads by rotating at 15 rpm at 4° C. in imidazole in 1× native resuspension buffer without Triton X-100.

Protein interaction studies were performed as follows: p97 was coupled to S -protein agarose (Merck Milipore) for 2 hours at 4° C. in buffer containing 137 mM NaCl, 20 mM Tris HC1 pH 7.0, then incubated TEX264WT or TEX264ΔSHP in the same buffer, supplemented with 0.5% Triton X-100. TEX264WT was coupled to HisPur™ Ni-NTA Magnetic Beads (Thermo Fisher), prior to incubation with recombinant Topoisomerase 1 (Inspiralis) in a buffer composed of 137 mM NaCl, 20 mM Tris HCl pH 7.0. Reactions were terminated by the addition of Laemmli sample buffer, and resolved by SDS-PAGE.

### Rapid Approach to DNA Adduct Recovery (RADAR)

DPCs were isolated using a modified RADAR assay (Kiianitsa and Maizels, 2013). Cells were grown to ˜70% confluency then lysed in M buffer (MB), containing 6 M guanidine thiocyanate, 10 mM Tris—HCl (pH 6.8), 20 mM EDTA, 4% Triton X-100, 1% N-lauroylsarcosine and 1% dithiothreitol. DNA was precipitated by adding 100% ethanol, then washed three times in wash buffer (20 mM Tris—HC1 pH 7.4, 150 mM NaCl and 50% ethanol), and solubilized in 8 mM NaOH. DNA concentrations were quantified by NanoDrop™ and confirmed by slot blot analysis on a Hybond N+membrane followed by detection with an anti-dsDNA antibody. For TOP1ccs, samples were digested with Benzonase for 30 minutes at 37° C. and analysed by slot blot analysis on a Nitrocellulose membrane.

### Colony Forming Assay

HeLa cells were seeded in triplicate on 6-well plates and allowed to attach for 16 hours. Cells were then treated with the indicated doses of CPT (Sigma-Aldrich) for 24 hours, after which they were washed with PBS, and released into normal media. Colonies were fixed and stained 7-10 days later, the number of colonies was counted using the automated colony counter, GelCount™ (DTI-Biotech). The number of colonies in treated samples is expressed as a percentage of the number of colonies in the untreated samples.

### SUMO Binding Assay

HEK293 cells transiently expressing TEX264-GFP, TEX264SIM1, or TEX264SIM2 were lysed in a lysis buffer containing 3% Triton X-100 and 1M NaCl. Samples were sonicated using a Bioruptor Plus sonicator (30 seconds ON, 30 seconds OFF for 3 cycles), then diluted (1:3 volume) in IP wash buffer (150 mM NaCl, 50 mM Tris-HC1; 0.5 mM EDTA) and incubated with GFP trap beads (Chromotek). After capture, the beads were washed 3 times in IP wash buffer and incubated with 1 μg of free SUMO1 or SUMO2 (Boston Biochem). After washing in 3× in IP wash buffer, the samples were eluted in Laemlli buffer for 5 minutes at 95° C.

### Isolation of Proteins on Nascent DNA (iPOND)

iPOND was performed as described previously (Sirbu et al, 2011), with the following modifications. HEK293 cells were incubated for 10 minutes with 10 μM EdU. TEX264-depleted cells were pulse-labelled with EdU for 20 minutes to account for the ˜2-fold reduction in DNA replication fork velocity in these cells. In thymidine chase experiments, cells were incubated with normal media supplemented with 10 μM thymidine. Chromatin was fragmented into 50-300 bp fragments by sonication with a Bioruptor Plus sonicator (30 seconds ON, 30 seconds OFF for 50 cycles). Biotin-labelled EdU was captured by incubating samples overnight with streptavidin-coupled agarose beads (Merck Millipore).

### DNA Fibre Assay

HEK293 cells were incubated in media containing 30 μM CldU (Sigma-Aldrich, C6891) for 30 min, washed 3 × in PBS, and then incubated with media containing 250 μM of IdU (Sigma-Aldrich, 17125) for an additional 30 min. Cells were then treated with ice-cold PBS. Cells were lysed in 200 mM Tris-HC1 pH 7.4, 50 mM EDTA and 0.5% SDS directly onto glass slides and then fixed with 3:1 methanol and acetic acid overnight at 4° C. The next day, the DNA fibres were denatured with 2.5 M HC1, blocked with 2% BSA and stained with antibodies that specifically recognise either CldU (Abcam, Ab6326, dilution 1:500) or IdU (BD-347580, dilution 1:500). Anti-rat Cy3 (dilution 1:300, Jackson Immuno Research, 712-116- 153) and anti-mouse Alexa-488 (dilution 1:300, Molecular Probes, A11001) were used as the respective secondary antibodies. Microscopy was performed using a Nikon 90i microscope. The lengths of the IdU-labelled tracts were measured using ImageJ software and converted into microns. Statistical analysis was done by GraphPad Prism software using an unpaired t-test.

### Immunofluorescence

Visualisation of TOP1ccs by immunofluorescence was performed as described by Patel et al, 2016. Briefly, cells were fixed in 4% formaldehyde for 15 mins at room temperature, then permeabilised in 0.5% Triton X-100 for 15 minutes at 4° C. Cells were then treated with 0.5% SDS for 5 minutes are room temperature and washed 5 times in a buffer containing 0.1% Triton X-100 and 0.1% BSA diluted in PBS. After blocking in 5% BSA/PBS for 1 hour at room temperature, cells were incubated with an anti-TOP1cc antibody (Merck) diluted 1:100 in 2.5% BSA/PBS. Following staining with secondary antibodies and DAPI, coverslips were mounted onto slides and imaged using a Nikon 90i microscope.

### In Vitro TOP1cc Repair Assay

FlpIn T-Rex HEK293 cells were induced with 1 mg/ml doxycycline for 48 hours to deplete TDP1, then treated with 10 μg/ml MG132 for 1 hour and 14 μM CPT for 30 minutes. Cells were lysed in a guanidine hydrochloride-based lysis buffer and separated by CsCl-gradient fractionation. TOP1ccs were then precipitated with ice cold 100% acetone at -80° C. for 30 minutes, washed with 70% ethanol, air dried, and resuspended. Samples were dialysed overnight at 4° C. and sedimented by centrifugation 15,000 x g for 20 minutes to remove aggregated proteins. Samples were incubated with the indicated proteins at 37° C., slot blotted onto a nitrocellulose membrane, and probed with a TOP1cc-specific antibody.

### Immunohistochemistry

The study protocol was in accordance with the ethical guidelines of the Helsinki declaration. Tissue samples were prepared from paraffin blocks according to standard histological protocols and immunohistochemical staining was performed using Leica Bond automated staining system. SPRTN (Atlas) and TEX264 (Novus) antibodies were diluted 1:1000. Appropriate IgG isotype—matched negative control antibodies were used in each case. Measurements for ‘strong’ positive staining were obtained using the Image Scope Positive Pixel Count v9 algorithm.

Ethics approval was granted by the National Research Ethics Service South Central Oxford—Panel C ethics committee (number 13/SC/0111). Regulatory approval was granted by the UK Medicines and Healthcare products Regulatory Agency (clinical trial authorisation [CTA] number 00316/0245/001-0001). All trial procedures and processes complied with the International Conference on Harmonisation's Good Clinical Practice guidelines. We asked patients to sign a consent form for registration and for analysis of the biomarker panel; additional written informed consent was obtained before randomisation in FOCUS4-D.

### Plasmid & siRNA Transfections

Plasmid DNA transfections were performed using polyethyleneimine (PEI) reagent, Lipofectamine 3000 (Thermo Fisher), or FuGENE HD Transfection Reagent (Promega), following the manufacturer's instructions.

All siRNA transfections were carried out using Lipofectamine RNAiMAX (Invitrogen), according to the manufacturer's protocol and assayed after 72 hours. siRNA sequences used in this study are provided in the materials table below.

### Materials

| | | |
|---|---|---|
| REAGENT or RESOURCE | SOURCE | IDENTIFIER |

Antibodies

| | | |
|---|---|---|
| Phospho-gammaH2AX (Ser139) | Genetex | Cat# GTX127342* |
| HA [clone 3F10] (rat monoclonal) | Roche | Cat# 3F10; RRID:AB_2314622 |
| PCNA (mouse monoclonal) | Abeam | Cat# ab29; RRID:AB_303394 |
| p97 (rabbit polyclonal) | Proteintech | Cat# 10736-1-AP, RRID:AB_2214635 |
| p97 (rabbit polyclonal) | In house | N/A |
| Topoisomerase 1 (rabbit polyclonal) | Bethyl Laboratories | Cat# A302-589A; RRID:AB_2034865 |
| Topoisomerase 1-DNA Covalent | Millipore | Cat# MABE1084; RRID:AB_2756354 |
| Complexes Antibody, clone 1.1 A | | |
| (mouse monoclonal) | | |
| TEX264 (rabbit polyclonal) | In house | N/A |
| TEX264 (mouse monoclonal) | Novus | Cat# H00051368-M01; |
| | | RRID:AB_2255844 |
| Vinculin (mouse monoclonal) | Bethyl Laboratories | Cat# ab18058; RRID:AB_444215 |
| Flag (rabbit polyclonal) | Sigma-Aldrich | Cat# F7425; RRID:AB_439687 |
| Myc (mouse monoclonal) | CellSignaling | Cat# 2276, RRID:AB_331783 |
| | Technology | |
| Double-stranded DNA (mouse | Abeam | Cat# ab27156; RRID:AB_470907 |
| monoclonal) | | |
| SUMO1 (rabbit polyclonal) | Abeam | Cat# ab11672, RRID:AB_298480 |
| SUMO2/3 , clone 18H8 (rabbit | CellSignaling | Cat# 4971, RRID:AB_2198425 |
| monoclonal) | Technology | |
| SPRTN N-terminal (rabbit polyclonal) | In house; Lessel | N/A |
| | et.al, 2014 | |
| SPRTN C-terminal (rabbit polyclonal) | Atlas | HPA025073 |
| GFP | Abeam | Cat# ab290, RRID:AB_303395 |
| POLD1 (mouse monoclonal) | Abeam | Cat# ab10362, RRID:AB_297099 |
| MCM7 (141.2) (mouse monoclonal) | Santa Cruz | Cat# sc-9966, RRID:AB_627235 |
| | Biotechnology | |
| Histone H3 (rabbit polyclonal) | Abeam | Cat# ab1791; RRID:AB_30261 3 |
| ds-DNA (mouse monoclonal) | Abeam | Cat# ab27156; RRID:AB_470907 |
| TDP1 (rabbit polyclonal) | Abeam | Cat# ab4166, RRID:AB_304337 |
| Mono- and polyubiquitinylated | Enzo Life Sciences | Cat# BML-PW8810, RRID:AB_10541840 |
| conjugates, mAb (FK2) | | |
| Tubulin (mouse monoclonal) | Sigma-Aldrich | Cat# T6199, RRID:AB_477583 |
| Histone H2A.X, phospho (Ser139; | Millipore | Cat# 05-636, RRID:AB_309864 |
| clone JBW301; mouse monoclonal) | | |
| Rabbit IgG, HRP-conjugated (goat | Sigma-Aldrich | Cat#A9169; RRID:AB_258434 |
| polyclonal) | | |
| Mouse IgG, HRP-conjugated (rabbit | Sigma-Aldrich | Cat#A9044; RRID:AB_258431 |
| polyclonal) | | |
| Mouse IgG, Alexa Fluor 488 (goat | Molecular Probes | Cat# A-11001; RRID:AB_2534069 |
| polyclonal) | | |
| Rabbit IgG, Alexa Fluor 594 (donkey | ThermoFisher | Cat# R37119, RRID:AB_2556547 |
| polyclonal) | Scientific | |
| Mouse IgG, Alexa Fluor 488 | ThermoFisher | Cat# R37114, RRID:AB_2556542 |
| | Scientific | |
| Rat IgG, Cy3-AffinityPureF(ab')2 | Jackson | Cat# 712-166-153 RRID:AB_2340669 |
| Fragment (donkey polyclonal) | ImmunoResearch | |
| | Labs | |

Bacterial and Virus Strains

| | | |
|---|---|---|
| Escherichia coli DH5a | Invitrogen | Cat# 18265-017 |
| Escherichia coli Rosetta 2 (DE3) | Novagen | Cat# 71405-3 |

Chemicals, Peptides, and Recombinant Proteins

| | | |
|---|---|---|
| Camptothecin | Calbiochem | Cat# 208925 |
| 5-Chloro-2’ deoxyuridine (CldU) | Sigma-Aldrich | Cat# C6891 |
| 5-lodo-2’-deoxyuridine (IdU) | Sigma-Aldrich | Cat# 17125 |
| 5-Ethynyl-2’-deoxyuridine (EdU) | Sigma-Aldrich | Cat# T511285 |
| 5-Bromo-2’-deoxyuridine (BrdU) | Sigma-Aldrich | Cat# B9285 |
| CB-5083 | BioVision | Cat# B1032 |
| Doxycycline | Panreac | Cat# A2951, 0025 |
| Accuprime Pfx DNA polymerase | Invitrogen | Cat# 12344-024 |
| FuGene HD | Promega | Cat# E2311 |
| Lipofectamine siRNAMax | Invitrogen | Cat# 13778-150 |
| Polyethylenimine | Sigma-Aldrich | Cat# 408727 |
| Benzonase | Millipore | Cat# 71205 |
| Human TEX264 protein | This study | N/A |
| Human p97 protein | This study | N/A |
| Human TDP1 protein | Sherif El-Khamisy | N/A |
| | Lab | |
| Human topoisomerase 1 protein | l nspiralis | Cat# HT101 |

Critical Commercial Assays

| | | |
|---|---|---|
| Clarity™ Western ECL Substrate | Bio-Rad | Cat# 1705061 |
| SuperSignal™ West Femto Maximum | ThermoFisher | Cat# 34095 |
| Sensitivity Substrate | Scientific | |
| SuperSignal™ West Pico PLUS | ThermoFisher | Cat# 34580 |
| Chemiluminescent Substrate | Scientific | |
| ProteoSilver Plus Silver Stain Kit | Sigma-Aldrich | Cat# PROTSIL2 |

Experimental Models: Cell Lines

| | | |
|---|---|---|
| HEK293 cells | ATCC | Cat# CRL-1573, RRID:CVCL_0045 |
| U-2 OS cells | ATCC | Cat# HTB-96, RRID:CVCL_0042 |
| HeLa cells | ATCC | Cat# CCL-2, RRID:CVCL_0030 |
| hTERT-RPE1 | ATCC | Cat#CRL-4000; RRID:CVCL_4388 |
| ΔTEX264 HEK293 cells | This study | N/A |
| HEK293-Flp-ln TRex TEX264-wt- | This study | N/A |
| cSSH | | |

Oligonucleotides

| | | |
|---|---|---|
| siLuc: CGUACGCGGAAUACUUCGA | This study | SEQ ID NO: 5 |
| sip97 #1: | This study | SEQ ID NO: 6 |
| AAGUAGGGUAUGAUGACAUUG | | |
| sip97 #2: | This study | SEQ ID NO: 7 |
| AAGAUGGAUCUCAUUGACCUA | | |
| sip97 #3: | This study | SEQ ID NO: 8 |
| AACAGCCAUUCUCAAACAGAA | | |
| siTEX264 #1: | This study | SEQ ID NO: 9 |
| CTCATCGACCTCTACCAGAAA | | |
| siTEX264 #2: | This study | SEQ ID NO: 10 |
| CGGCTGGAGATCTACCAGGAA | | |
| siTEX264 3’UTR: | This study | SEQ ID NO: 11 |
| CUUCCAGGACCCAGAAUAA; | | |
| siSPRTN #1: | Lessel et al., 2014 | SEQ ID NO: 12 |
| CACGAUGAGGUGGAUGAGUAU; | | |
| siTOP1: | Vaz et al., 2016 | SEQ ID NO: 13 |
| GGUCCCUGUUGAGAAACGA | | |
| siTDP1: | This study | SEQ ID NO: 14 |
| GGAUAUUGCGUUUGGAACA | | |

Recombinant DNA

| | | |
|---|---|---|
| pCDNA5-FRT/TO-cSSH | Vaz et al., 2016 | N/A |
| pCDNA5-FRT/TO-SPRTN-wt-cSSH | Vaz et al., 2016 | N/A |
| pOTB7 TEX264 cDNA Clone | Source Bioscience | N/A |
| IRAUp969H0427D | | |
| pGEX-4t-2 TEX264 nGST | This study | N/A |
| pCDNA5 FRT/TO Flpln cSSH | ThermoFisher | N/A |
| | Scientific | |
| pET21a(+) TEX264 NTA cHis | This study | N/A |
| pET21a(+) TEX264 NTA SHPA cHis | This study | N/A |
| pCDNA5 FRT/TO Flpln TEX264 | This study | N/A |
| cSSH | | |
| pCDNA3.1 TEX264-FLAG | This study | N/A |
| pCDNA5 FRT/TO TEX264-GFP | This study | N/A |
| pET21a(+) p97-S*Tag | This study | N/A |
| pCDNA5 FRT/TO p97-Myc-Strep | This study | N/A |
| pNIC-ZB-SPRTN | Vaz et al., 2016 | N/A |
| YFP-TOP1 | Gift from Sherif El- | N/A |
| | Khamisy | |
| pCI-Myc-TDP1 | Gift from Sherif El- | N/A |
| | Khamisy | |

Software and Algorithms

| | | |
|---|---|---|
| Graphpad Prism v6.01 | Graphpad Software | http://www.graphpad.com/ |
| ImageLab v5.2.1 | Bio-Rad | http://www bio-rad.com/en- |
| | Laboratories | ch/produet/image-lab-sofware |
| Imaged 1.48v | Wayne Rasband | https://imagei.nih.gov/ii/ |
| | (NIH) | |
| Aperio ImageScope [v12.3.2.801 3] | Leica Biosystems | https://www.leicablosystems. |
| | | com/diaital-pathology/manage/ |
| | | aperio-imagescope/ |

### Example 1 - The ATPase p97 is Involved in TOP1cc Repair

To identify modulators of TOP1cc repair in human cells, chromatin was isolated from YFP-TOP1-expressing human embryonic kidney (HEK) 293 cells and subjected YFP immunoprecipitates to liquid chromatography-tandem mass spectrometry (LC MS/MS). This analysis identified the AAA ATPase p97 as an abundant interacting partner of TOP1 on chromatin, this was confirmed by immunoblotting (FIG. 7). By using energy generated from ATP hydrolysis, p97 is able to remodel its substrates and extract them from macromolecular structures such as chromatin (Bodnar and Rapoport, 2017a, 2017b). Given this known role of p97, and since Cdc48 has been implicated in TOP1-expressing repair, whether p97 contributes to TOP1cc processing in human cells was investigaed.

To assess if p97-deficient cells accumulate basal TOP1ccs, modified version of the recently described RADAR (Rapid Approach to DNA Adduct Recovery) assay was employed to analyse the abundance of proteins covalently attached to DNA (Kiianitsa and Maizels, 2013). By lysing cells in chaotropic salts (6M guanidium isothiocyanate), detergents (4% Triton X-100 and 1% N-lauroylsarcosine), and a reducing agent (1% DTT), all molecular interactions, other than covalent interactions, are disrupted. Depletion of p97 in HEK293 cells with two different siRNA sequences resulted in substantial TOP1cc accumulation, to a similar extent as a short treatment with 1 μM CPT (FIG. 1A, B, C). Mechanistically, p97 forms a hexamer and uses energy generated by ATP hydrolysis to remodel its substrates by threading them through its central pore (Blythe et al., 2017; Bodnar and Rapoport, 2017b). TOP1 bound more strongly to a substrate-trapping, ATPase-defective p97 mutant (E578Q) than wild-type p97 (FIG. 1D). This suggested that the ATPase activity of p97 could be required to counteract TOP1cc accumulation. To test this human retinal pigmented epithelial (RPE-1) cells were treated with CB-5083, a potent, selective inhibitor of p97 ATPase activity, and monitored TOP1cc foci formation by immunofluorescence using an antibody which specifically recognises TOP1ccs (FIG. 1E) (Anderson et al., 2015; Patel et al., 2016).

Acute p97 inhibition resulted in TOP1cc accumulation (FIG. 1F, G). Overall, it was concluded that p97 ATPase activity is needed to counteract TOP1cc accumulation in vivo, as is the case for Cdc48 (Ruggiano et al., 2016; Stingele et al., 2014).

### Example 2 - TEX264 is a p97 Cofactor and Recruits p97 to TOP1

To recognise and process its diverse substrates, p97 associates with cofactors which directly bind to p97 via conserved p97-interaction motifs, and typically bridge p97 to ubiquitinated substrates through ubiquitin-binding domains (Meyer, et al., 2012). To identify the p97 cofactor that targets p97 to TOP1ccs , an ongoing mass spectrometry screen of proteins that interact with p97 in the nucleus was consulted (unpublished data). A protein which stood out as a potential candidate was the uncharacterised protein Testes-expressed 264 (TEX264; Q9Y619) because it possesses a gyrase inhibitory-like (Gyrl-like) domain (FIG. 2A). In E. coli, Gyrl-like proteins have been shown to inhibit the decatenation activity of the bacterial type II topoisomerase, DNA gyrase (Na kanishi, et al., 1998; Sengupta & Nagaraja, 2008). On closer analysis of the TEX264 protein sequence a putative p97 interaction motif, known as a SHP box, was identified located in its C-terminus, suggesting that TEX264 could be a p97 cofactor (FIG. 2A, B). Orthologs of TEX264 are present in vertebrates, including teleost fish, but are absent in established model organisms such as S. cerevisiae, S. pombe, and C. elegans.

To address whether TEX264 is indeed a p97 cofactor, human p97, wild type TEX264 (TEX264WT) and a TEX264 mutant lacking its putative SHP box (TEX264ΔSHP; amino acids 273-285) were purified from bacteria. TEX264WT readily bound p97 but TEX264ΔSHP did not, establishing TEX264 as a novel p97 cofactor (FIG. 2C). Attempts were made to reconstitute the p97-TEX264-TOP1 complex in vitro. TEX264WT could efficiently associate with recombinant TOP1, whereas direct binding between p97 and TOP1 was either weak or not detected (FIG. 7B, C, D). However, when p97 was incubated with TEX264 prior to the addition of TOP1, a robust increase in the amount of TOP1 in p97 pulldowns (FIG. 7D, E) was observed. This demonstrates that TEX264 can simultaneously bind both p97 and TOP1 and, thus, physically bridge p97 to TOP1. TEX264 is present on chromatin and also forms a complex with TOP1 and p97 in vivo (FIG. 7F, G & FIG. 2D). The interaction between TEX264 and TOP1 increased markedly upon treatment with CPT, indicating that TEX264 is recruited to TOP1ccs, along with p97 (FIG. 2D & FIG. 7G). TOP1 was readily detectable in p97 immunoprecipitates prepared from wild-type cells but was only faintly detectable in those prepared from CRISPR- Cas9 TEX264 knockout cells (ΔTEX264), confirming that TEX264 is required to recruit p97 to TOP1 in vivo (FIG. 2E). These interactions were resistant to benzonase and ethidium bromide, indicating that they are not mediated by DNA. It was noted that TOP1 did not co-immunoprecipitate with either p97 or TEX264 after treatment with 1 μM CPT (FIG. 2E & FIG. 7G). This dose causes significant double strand break formation and replication fork collapse and far exceeds clinically-relevant doses (which are in the nanomolar range) (Ray Chaudhuri et al., 2012; Takimoto and Arbuck, 1997). Together, these data establish TEX264 as a novel p97 cofactor which is recruited to TOP1ccs and bridges p97 to TOP1 both in vivo and in vitro.

### Example 3 - TEX264 Promotes TOP1cc Repair and is Epistatic with p97 and TDP1

Depletion of TEX264 resulted in significant TOP1cc foci accumulation in RPE-1 and U-2 osteosarcoma (U205) cells (FIG. 8A, B & FIG. 3G, H). Knockout of TEX264 also caused substantial TOP1cc accumulation in HEK293 cells (FIG. 3A & FIG. 8C). This was specifically due to loss of TEX264 as expression of exogenous TEX264 in ΔTEX264 cells could completely reverse this increase (FIG. 8D). However, exogenous TEX264 could not reverse TOP1cc accumulation when ΔTEX264 cells were depleted of p97, revealing that TEX264 requires p97 to counteract TOP1cc accumulation (FIG. 8D). Following a short CPT treatment and release into CPT-free media, TOP1ccs were rapidly resolved in control cells, but persisted long after CPT withdrawal in ΔTEX264 cells. This indicates that ΔTEX264 cells have a TOP1cc repair defect (FIG. 3B, C). An assessment was then made to determine whether TEX264 and TDP1 are epistatic. Depletion of TEX264, TDP1, or p97 resulted in similar increases in basal TOP1cc accumulation (FIG. 8E, F, G). Depleting each factor in combination did not result in any further increase in TOP1cc accumulation. This indicates that TEX264, p97, and TDP1 act in the same pathway to repair TOP1ccs. In further support of this, TEX264- depleted cells exhibited hyper-sensitivity to low doses of CPT, which was not further enhanced upon TDP1 depletion (FIG. 3D). Notably, TEX264-depleted cells expressing exogenous TDP1 were as sensitive to CPT as TEX264-depleted cells alone, suggesting that TDP1 requires TEX264 to repair TOP1ccs (FIG. 8H, I). The crystal structure of the bacterial Gyrl-like protein, SbmC, revealed that the protein forms a solvent-exposed surface which may mediate substrate binding (Romanowski, et 1., 2002). Based on this information, TEX264 variants with single point mutations in conserved residues in or close to its Gyrl-like domain were generated and their ability to bind TOP1 tested (FIG. 3E). Each variant displayed reduced binding to TOP1, suggesting that they comprise a binding surface that enables TEX264 to bind TOP1 (FIG. 3F). When TEX264 expression was suppressed using siRNA targeting its 3′ UTR, U2OS cells accumulated approximately 3-fold more TOP1cc foci. Wild-type TEX264 could completely reverse this increase, whereas the TOP1 binding-defective variant, E194A, failed to do so (FIG. 3G, H). These results demonstrate that the interaction between TEX264 and TOP1 is important for its role in counteracting TOP1cc formation.

### Example 4 - TEX264 Recognises SUMOylated TOP1ccs

Most p97 cofactors have ubiquitin-binding domains that direct p97 to ubiquitinated proteins (Meyer, et al., 2012). TEX264, however, does not appear to contain ubiquitin-binding motifs, nor could direct binding between TEX264 and poly-ubiquitin chains be detected (data not shown). As SUMOylation of TOP1ccs is proposed to facilitate their repair, and Cdc48 has been shown to act on SUMOylated substrates, such as Rad52 and TOP1, investigation were carried out to look at whether TEX264 is linked to SUMO-mediated TOP1cc repair (Bergink et al., 2013; Heideker et al., 2011; Mao et al., 2000; Nie et al., 2012). Thus far, no SUMOylated substrates of p97 have been identified in metazoans.

Short treatment with a low dose of CPT induced moderate increases in SUMOylated TOP1, particularly SUMO1, which might serve as a signal for the recruitment of TOP1cc repair factors (FIG. 9a). In the absence of these repair factors, SUMOylated TOP1 would therefore be expected to accumulate. Indeed, depletion of TEX264 resulted in substantial increases in SUMOylated as well as ubiquitinated forms of TOP1 (FIG. 9b). Bioinformatic analysis subsequently revealed the presence of two putative SUMO-interacting motifs (SIMs) located in the Gyrl-like domain of TEX264 (FIG. 4a). Therefore tests were carried out to determine whether TEX264 directly interacts with SUMO. Purified TEX264 bound to free SUMO1, but not SUMO2 (neither free SUMO2 nor poly-SUMO2 chains; FIG. 4b & FIG. 9c). Mutation of either of the putative SIMs strongly diminished SUMO1 binding (FIG. 4b). Co-immunoprecipitation experiments revealed that both SIM mutants pulled-down less SUMOylated TOP1 than wild-type TEX264, but only the SIM2 mutant displayed reduced overall TOP1 binding, probably because it binds even less efficiently to SUMO1 than the SIM1 mutant (FIG. 4c, d). In turn, TEX264WT and TEX264SIM1 could reverse TOP1cc accumulation in TEX264-depleted U2OS cells, whereas TEX264SIM2 displayed a strongly reduced ability to do so (FIG. 4e, f). It was concluded that SUMO1 and SIM2 of TEX264 facilitate the recruitment of TEX264 to TOP1ccs in vivo to promote their repair.

### Example 5- TEX264 Promotes SPRTN-Dependent TOP1cc Repair

In principle, TEX264 and p97 might together be capable of recognising and remodelling TOP1ccs so as to facilitate access of TDP1 to the phosphodiester bond that links TOP1 to DNA. Whether other factors contribute in vivo remained unclear. It was recently demonstrated that another p97 cofactor, SPRTN, is a metalloprotease which can proteolyse TOP1, amongst other DNA-protein crosslinks (DPCs) during DNA replication (Lopez-Mosqueda et al., 2016; Maskey et al., 2017; Morocz et al., 2016; Stingele et al., 2016; Vaz et al., 2016). To assess the interplay of TEX264 and SPRTN, both proteins were depleted in HeLa cells, either alone or in combination, and assessed cellular sensitivity to CPT. Depletion of SPRTN alone sensitised cells to CPT, albeit to a lesser extent than TEX264 depletion (FIG. 10a, b). However, co-depletion of SPRTN did not further sensitise TEX264-depleted cells, indicating that these proteins can co-operate to repair TOP1ccs but also that TEX264 has SPRTN-independent roles in counteracting TOP1cc-induced cytotoxicity.

To explore how TEX264 regulates SPRTN-dependent TOP1cc processing, SPRTN-SSH was immunoprecipitated from wild-type and TEX264-depleted HEK293 cell extracts. SPRTN-SSH co-immunoprecipitated with endogenous TOP1, p97, and TEX264 in wild-type cell extract, however depletion of TEX264 strongly reduced the interaction between SPRTN and TOP1 without affecting total TOP1 levels or the interaction between SPRTN and p97 (FIG. 10c). As p97 exists in hexameric complexes, it can bind multiple cofactors at a time (Buchberger et al., 2015; Hänzelmann et al., 2011). The data presented here indicates that TEX264 recruits p97-SPRTN sub-complexes to TOP1ccs. Interestingly, unlike SPRTN, TEX264 inactivation did not result in the accumulation of total DPCs, suggesting that TEX264 recruits SPRTN specifically to TOP1ccs, and not all SPRTN substrates (FIG. 10d, e). As SPRTN preferentially cleaves disordered protein regions, it was hypothesised that p97 might remodel TOP1ccs to expose disordered regions which are more amenable to SPRTN-dependent cleavage (Vaz et al., 2016). To test this model, an in vitro assay was performed to assess the ability of SPRTN to process TOP1ccs isolated from cells by CsCl-gradient fractionation. SPRTN alone could process only a small proportion of TOP1ccs. However, this activity was enhanced when TOP1ccs were pre-incubated with p97 and TEX264 prior to the addition of SPRTN (FIG. 10f).

### Example 6 - TEX264 and p97 Act at Replication Forks

SPRTN is a replication-coupled DPC repair protein that also recruits p97 to stalled replication forks (Davis et al., 2012; Duxin et al., 2014; Larsen et al., 2018; Mosbech et al., 2012; Vaz et al., 2016). Based on this and the fact that TOP1ccs can stall DNA replication, the question is whether TEX264 and p97 also act near replication forks to prevent TOP1ccs from impeding fork progression. In addition to TOP1, mass spectrometry analysis of anti-HA chromatin immunoprecipitates isolated from HEK293 cells stably expressing TEX264-SSH detected numerous replisome components, including the entire MCM complex and PCNA. We confirmed that stably-expressed TEX264 exists in a complex with many components of the replisome (FIG. 5a). In support of TEX264 and p97 playing a role in DNA replication, both proteins were detected at replication forks by iPOND (isolation of proteins on nascent DNA; FIG. 5b, c) (Sirbu et al., 2011). Furthermore, measurement of DNA replication fork speed by DNA fibre assay, revealed that loss of TEX264 or p97 caused DNA replication forks to progress more slowly (FIG. 11a).

Depletion of TEX264 resulted in a strong enrichment of TOP1 at replication forks, which likely reflects the failure of these cells to repair TOP1ccs (FIG. 5d). It was reasoned that reducing the prevalence of replication-blocking TOP1ccs should alleviate the replication fork defects observed in TEX264-depleted cells. Strikingly, depletion of TOP1 in TEX264-deficient cells restored DNA replication fork velocity to that observed in control cells (FIG. 5e). Moreover, TOP1 depletion almost completely alleviated DNA strand break accumulation in TEX264-deficient cells, as measured by γH2AX (FIG. 11b, c, d). Thus, the replication defects and DNA strand breaks observed in TEX264-deficient cells can, largely, be attributed to the deleterious action of the TOP1 protein and the consequent formation of TOP1ccs.

### Example 7- High SPRTN Expression Correlates with Irinotecan Resistance in Metastatic Colorectal Cancers

The FOCUS clinical trial was initiated to assess whether the camptothecin derivative, irinotecan, could improve the prognosis of patients with metastatic colorectal cancer. Treatment with fluorouracil (FU) and irinotecan was found to improve patient response rates to 40-50%, versus 10-15% when only FU was administered. However, up to 60% of patients still did not respond to therapy, underscoring the importance of identifying molecular correlates of resistance to improve patient stratification (Seymour et al., 2007).

It was speculated that, based on its role in resolving TOP1ccs, the p97-SPRTN-TEX264 complex could impact the clinical efficacy of TOP1 poisons. Specifically, it was hypothesised that patients with tumours expressing high levels of TEX264 and/or SPRTN would be less likely to respond to the therapeutic TOP1 poison, irinotecan. To test this hypothesis, access to primary tumour material retrieved from 83 patients who went on to receive FU plus irinotecan (FOLFIRI) in the national FOCUS trial (Seymour et al, 2007) was obtained. Using the trial data, patients 6 were selected who achieved a complete or partial response to FOLFIRI, and 6 who did not (1 stable disease; 5 progressive disease). TEX264 and SPRTN expression was assessed in these 12 patients by immunohistochemistry (FIG. 6 & FIG. 12). It was observed that while TEX264 was highly expressed in all tested samples, SPRTN expression was more broad and intense in the cancers biopsied from patients with progressive or stable disease (FIG. 6a, b, d & FIG. 12a, b, d). The correlation between high SPRTN expression and poor response to irinotecan demonstrates that SPRTN expression may be used as a biomarker of irinotecan resistance in metastatic colorectal cancer.

### DISCUSSION

TOP1ccs are highly cytotoxic and clinically-relevant DNA lesions. Much effort has been placed on identifying factors that repair TOP1ccs as it is anticipated that targetting such factors could enhance the clinical efficacy of TOP1 poisons and/or overcome drug resistance (Pommier, 2006). The data presented herein elucidates a key aspect of the TOP1cc repair process, specifically how TOP1ccs are processed upstream of the phosphodiesterase TDP1. The bulky nature of the TOP1 protein restricts TDP1′s access to the phosphodiester bond that links TOP1 to DNA. It has long been appreciated that heat denaturation or pre-digestion of a TOP1cc with trypsin enables TDP1 activity in vitro, however, a detailed understanding of TOP1cc processing upstream of TDP1 in vivo has been lacking.

The results presented here demonstrate that the ATPase p97 and metalloprotease SPRTN act with the hitherto uncharacterised protein TEX264 to repair TOP1ccs. TEX264 binds p97 via a SHP box and recruits it to TOP1. TEX264 possesses SIMs that enable it to interact with SUMO1 and SUMOylated TOP1. This, in turn, facilitates and/or stabilises direct binding between the Gyrl-like domain of TEX264 with TOP1. The data indicates that the ATPase activity of p97 is also required to process TOP1ccs. It is proposed that p97 remodels TOP1ccs to enable them to be proteolytically digested by the metalloprotease SPRTN. Once the bulk of the protein component of a TOP1cc is removed, the remaining DNA-bound peptide is excised by TDP1.

In yeast, a role for Cdc48/p97 and its cofactor, Ufdl, in the repair of SUMOylated TOP1ccs has been described. While the same SIM in Ufdl required for TOP1cc repair in yeast is not conserved in human Ufdl and the data presented here shows that TEX264 is required to recruit p97 to TOP1, a role for Ufdl-Np14 in TOP1cc repair in human cells is not ruled out. For instance, p97 hexamers can bind multiple cofactors in a hierarchical manner, as described for Ufd-Np14 and FAF1 (Hänzelmann et al., 2011). In this model, additional cofactor binding, such as by FAF1 (or TEX264), can provide an additonal layer of substrate-specificity control to p97-Ufd1-Np14 complexes.

The Cdc48/p97 cofactors and metalloproteases, Wss 1 (in yeast) and SPRTN (in metazoans), can digest the bulk of the protein component of TOP1ccs (Stingele, et al., 2014; Vaz, et al., 2016). The function of Wss1 in TOP1cc repair depends on Cdc48 but the reasons for this were unclear. It was speculated that Cdc48 could be involved in the removal of peptide remnants generated by proteolysis or in remodelling the TOP1 protein to facilitate its proteolytic digestion (Stingele, et al., 2014). The data presented here now suggests that proteolysis, at least by SPRTN, likely acts post-p97-mediated TOP1cc remodelling. It has previously been shown that SPRTN preferentially cleaves unstructured protein regions (Vaz et al., 2016). In contrast to other SPRTN substrates, such as histones, the TOP1 protein is large and lacking in unstructured regions. In line with this, in previous studies, it was shown that SPRTN cleaves TOP1 much less efficiently than histones in vitro. Therefore, the requirement of TEX264 and p97 in TOP1cc repair likely reflects the need to recognise and remodel the TOP1 protein to expose protein regions which are more amenable to cleavage by SPRTN.

It is important to note that SPRTN and Wssl are not homologs and this is reflected in differences in their cellular functions (Vaz et al, 2017, Fielden et al, 2018). For example, SPRTN appears to act in the same TOP1cc repair pathway as TDP1, whereas Wssl (and indeed Cdc48) acts in a parallel pathway to TDP1 in yeast (Balakirev et al., 2015; Nie et al., 2012; Stingele et al., 2014; Vaz et al., 2016). Specifically, yeast cells deficient in Wssl do not accumulate basal TOP1ccs or exhibit CPT sensitivity unless TDP1 is co-deleted and vice versa. In metazoans, loss of TDP1 alone results in substantial TOP1cc accumulation and sensitivity to TOP1 poisons (El-Khamisy et al., 2005; Katyal et al., 2014). The reasons for these differences are unclear but, fundamentally, reveal that there is a greater dependency on TDP1-mediated TOP1cc repair in mammalian cells than in yeast. Nevertheless, the TOP1 protein requires processing upstream of TDP1 and our results are consistent with the notion that TEX264, SPRTN, and p97 facilitate this process.

The data also suggests a new mode of SPRTN recruitment to specific DPCs; one which is mediated by a distinct p97 cofactor. Little is known about how SPRTN is recruited to, and recognises, specific DPCs. While SPRTN is recruited to stalled replication forks in a manner that requires its ability to bind PCNA and ubiquitin, some recent evidence indicates that these domains are not essential for DPC repair (Centore et al., 2012; Davis et al., 2012; Ghosal et al., 2012; Machida et al., 2012; Maskey et al., 2014, 2017; Mosbech et al., 2012; Stingele et al., 2016). As loss of TEX264 does not impair the repair of total DPCs, it is likely that TEX264 specifically recruits SPRTN to TOP1ccs.

Mutations in SPRTN cause early-onset hepatocellular carcinoma and premature ageing in humans. Hypomorphic SPRTN mice also manifest ageing phenotypes and develop liver tumours. These findings demonstrate the consequences of the genetic instability that arises when SPRTN function is disrupted. The data presented here that SPRTN expression positively correlates with irinotecan-resistance in colorectal cancer indicates that SPRTN can support cancer cell proliferation, particularly in the presence of DPC-inducing agents. This raises the possibility that the p97 system could be a potential target for chemotherapeutic intervention in these patients. While inhibitors of its ATPase activity are currently in clinical trials, p97 has broader roles in DNA repair and many other cellullar processes (Meerang, et al., 2011).

### REFERENCES

Anderson, D.J., Le Moigne, R., Djakovic, S., Kumar, B., Rice, J., Wong, S., Wang, J., Yao, B., Valle, E., Kiss von Soly, S., et al. (2015). Targeting the AAA ATPase p97 as an Approach to Treat Cancer through Disruption of Protein Homeostasis. Cancer Cell 28, 653-665.

Balakirev, M.Y., Mullally, J.E., Favier, A., Assard, N., Sulpice, E., Lindsey, D.F., Rulina, A. V., Gidrol, X., and Wilkinson, K.D. (2015). Wssl metalloprotease partners with Cdc48/Doal in processing genotoxic SUMO conjugates. Elife 4. e06763.

Bergink, S., Ammon, T., Kern, M., Schermelleh, L., Leonhardt, H., and Jentsch, S. (2013). Role of Cdc48/p97 as a SUMO-targeted segregase curbing Rad51-Rad52 interaction. Nat. Cell Biol. 15, 526-532.

Blythe, E.E., Olson, K.C., Chau, V., and Deshaies, R.J. (2017). Ubiquitin- and ATP-dependent unfoldase activity of P97/VCPNPLOC4UFD1L is enhanced by a mutation that causes multisystem proteinopathy. Proc. Natl. Acad. Sci. 114, E4380—E4388.

Bodnar, N., and Rapoport, T. (2017a). Toward an understanding of the Cdc48/p97 ATPase. F1000Research 6, 1318.

Bodnar, N.O., and Rapoport, T.A. (2017b). Molecular Mechanism of Substrate Processing by the Cdc48 ATPase Complex. Cell 169, 722-735.e9.

Buchberger, A., Schindelin, H., and Hänzelmann, P. (2015). Control of p97 function by cofactor binding. FEBS Lett. 589, 2578-2589.

Centore, R.C., Yazinski, S.A., Tse, A., and Zou, L. (2012). Spartan/Clorf124, a Reader of PCNA Ubiquitylation and a Regulator of UV-Induced DNA Damage Response. Mol. Cell 46, 625-635.

Davis, E.J., Lachaud, C., Appleton, P., MacArtney, T.J., Näthke, I., and Rouse, J. (2012). DVC1 (Clorf124) recruits the p97 protein segregase to sites of DNA damage. Nat. Struct. Mol. Biol. 19, 1093-1100.

Debéthune, L., and Kohlhagen, G. (2002). Processing of nucleopeptides mimicking the topoisomerase I—DNA covalent complex by tyrosyl-DNA phosphodiesterase. Nucleic Acids Res. 30, 1198-1204.

Duxin, J.P., Dewar, J.M., Yardimci, H., and Walter, J.C. (2014). Repair of a DNA-protein crosslink by replication-coupled proteolysis. Cell 159, 349-357.

El-Khamisy, S.F., Saifi, G.M., Weinfeld, M., Johansson, F., Helleday, T., Lupski, J.R., and Caldecott, K.W. (2005). Defective DNA single-strand break repair in spinocerebellar ataxia with axonal neuropathy-1. Nature 434, 108-113.

Fielden, J., Ruggiano, A., Popović, M., and Ramadan, K. (2018). DNA protein crosslink proteolysis repair: From yeast to premature ageing and cancer in humans. DNA Repair (Amst). 71, 198-204.

Ghosal, G., Leung, J.W.C., Nair, B.C., Fong, K.W., and Chen, J. (2012). Proliferating Cell Nuclear Antigen (PCNA)-binding protein Clorf124 is a regulator of translesion synthesis. J. Biol. Chem. 287, 34225-34233.

Hanzelmann, P., Buchberger, A., and Schindelin, H. (2011). Hierarchical binding of cofactors to the AAA ATPase p97. Structure 19, 833-843.

Heideker, J., Prudden, J., Perry, J.J.P., Tainer, J.A., and Boddy, M.N. (2011). SUMO-targeted ubiquitin ligase, Rad60, and Nse2 SUMO ligase suppress spontaneous Topl-mediated DNA damage and genome instability. PLoS Genet. 7. e1001320.

Interthal, H., Chen, H.J., and Champoux, J.J. (2005). Human Tdpl cleaves a broad spectrum of substrates, including phosphoamide linkages. J. Biol. Chem. 280, 36518-36528.

Katyal, S., Lee, Y., Nitiss, K.C., Downing, S.M., Li, Y., Shimada, M., Zhao, J., Russell, H.R., Petrini, J.H.J., Nitiss, J.L., et al. (2014). Aberrant topoisomerase-1 DNA lesions are pathogenic in neurodegenerative genome instability syndromes. Nat. Neurosci. 17, 813-821.

Kiianitsa, K., and Maizels, N. (2013). A rapid and sensitive assay for DNA-protein covalent complexes in living cells. Nucleic Acids Res. 41. e104.

Larsen, N.B., Gao, A.O., Sparks, J.L., Gallina, I., Wu, R.A., Mann, M., Ra \ddot{s}chle, M., Walter, J.C., and Duxin, J.P. (2018). Replication-Coupled DNA-Protein Crosslink Repair by SPRTN and the Proteasome in Xenopus Egg Extracts. Mol Cell 73(3):574-588.e7.

Lessel, D., Vaz, B., Halder, S., Lockhart, P.J., Marinovic-Terzic, I., Lopez-Mosqueda, J., Philipp, M., Sim, J.C.H., Smith, K.R., Oehler, J., et al. (2014). Mutations in SPRTN cause early onset hepatocellular carcinoma, genomic instability and progeroid features. Nat. Genet. 46, 1239-1244.

Lin, C.P., Ban, Y., Lyu, Y.L., Desai, S.D., and Liu, L.F. (2008). A ubiquitin-proteasome pathway for the repair of topoisomerase I-DNA covalent complexes. J. Biol. Chem. 283, 21074-21083.

Lopez-Mosqueda, J., Maddi, K., Prgomet, S., Kalayil, S., Marinovic-Terzic, I., Terzic, J., and Dikic, I. (2016). SPRTN is a mammalian DNA-binding metalloprotease that resolves DNA-protein crosslinks. Elife 5. e21491.

Machida, Y., Kim, M.S., and Machida, Y.J. (2012). Spartan/Clorf124 is important to prevent UV-induced mutagenesis. Cell Cycle 11, 3395-3402.

Mao, Y., Sun, M., Desai, S.D., and Liu, L.F. (2000). SUMO-1 conjugation to topoisomerase I: A possible repair response to topoisomerase-mediated DNA damage. Proc. Natl. Acad. Sci. U. S. A. 97, 4046-4051.

Maskey, R.S., Kim, M.S., Baker, D.J., Childs, B., Malureanu, L.A., Jeganathan, K.B., Machida, Y., Van Deursen, J.M., and Machida, Y.J. (2014). Spartan deficiency causes genomic instability and progeroid phenotypes. Nat. Commun. 5. 5744.

Maskey, R.S., Flatten, K.S., Sieben, C.J., Peterson, K.L., Baker, D.J., Nam, H.J., Kim, M.S., Smyrk, T.C., Kojima, Y., Machida, Y., et al. (2017). Spartan deficiency causes accumulation of Topoisomerase 1 cleavage complexes and tumorigenesis. Nucleic Acids Res. 45, 4564-4576.

Meyer, H., Bug, M., and Bremer, S. (2012). Emerging functions of the VCP/p97 AAA-ATPase in the ubiquitin system. Nat. Cell Biol. 14, 117-123.

Mórocz, M., Zsigmond, E., T6th, R., Zs Enyedi, M., Pinter, L., and Haracska, L. (2016). DNA-dependent protease activity of human Spartan facilitates replication of DNA-protein crosslink-containing DNA. Nucleic Acids Res. 45, 3172-3188.

Mosbech, A., Gibbs-Seymour, I., Kagias, K., Thorslund, T., Beli, P., Povlsen, L., Nielsen, S.V., Smedegaard, S., Sedgwick, G., Lukas, C., et al. (2012). DVC1 (Clorf124) is a DNA damage-targeting p97 adaptor that promotes ubiquitin-dependent responses to replication blocks. Nat. Struct. Mol. Biol. 19, 1084-1092.

Nakanishi, A., Oshida, T., Matsushita, T., Imajoh-Ohmi, S., and Ohnuki, T. (1998). Identification of DNA gyrase inhibitor (Gyrl) in Escherichia coli. J. Biol. Chem. 273, 1933-1938.

Nie, M., Aslanian, A., Prudden, J., Heideker, J., Vashisht, A.A., Wohlschlegel, J.A., Yates, J.R., and Boddy, M.N. (2012). Dual recruitment of Cdc48 (p97)-Ufdl-Np14 ubiquitin-selective segregase by small ubiquitin-like modifier protein (SUMO) and ubiquitin in SUMO-targeted ubiquitin ligase-mediated genome stability functions. J. Biol. Chem. 287, 29610-29619.

Patel, A.G., Flatten, K.S., Peterson, K.L., Beito, T.G., Schneider, P.A., Perkins, A.L., Harki, D.A., and Kaufmann, S.H. (2016). Immunodetection of human topoisomerase I-DNA covalent complexes. Nucleic Acids Res. 44, 2816-2826.

Pommier, Y. (2013). Drugging topoisomerases: Lessons and Challenges. ACS Chem. Biol. 8, 82-95.

Ray Chaudhuri, A., Hashimoto, Y., Herrador, R., Neelsen, K.J., Fachinetti, D., Bermejo, R., Cocito, A., Costanzo, V., and Lopes, M. (2012). Topoisomerase i poisoning results in PARP- mediated replication fork reversal. Nat. Struct. Mol. Biol. 19, 417-423.

Reardon, J.T., Cheng, Y., and Samar, A. (2006). Repair of DNA-protein cross-links in mammalian cells. Cell Cycle 5, 1366-1370.

Ruggiano, A., Mora, G., Buxo, L., and Carvalho, P. (2016). Spatial control of lipid droplet proteins by the ERAD ubiquitin ligase Doa10. EMBO J. 35, 1644-1655.

Sengupta, S., and Nagaraja, V. (2008). YacG from Escherichia coli is a specific endogenous inhibitor of DNA gyrase. Nucleic Acids Res. 36, 4310-4316.

Seymour, M.T., Maughan, T.S., Ledermann, J.A., Topham, C., James, R., Gwyther, S.J., Smith, D.B., Shepherd, S., Maraveyas, A., Ferry, D.R., et al. (2007). Different strategies of sequential and combination chemotherapy for patients with poor prognosis advanced colorectal cancer (MRC FOCUS): a randomised controlled trial. Lancet 370, 143-152.

Sirbu, B.M., Couch, F.B., Feigerle, J.T., Bhaskara, S., Hiebert, SM., and Cortez, D. (2011). Analysis of protein dynamics at active, stalled, and collapsed replication forks. Genes Dev. 25, 1320-1327.

Stingele, J., Schwarz, M.S., Bloemeke, N., Wolf, P.G., and Jentsch, S. (2014). A DNA-dependent protease involved in DNA-protein crosslink repair. Cell 158, 327-338.

Stingele, J., Bellelli, R., Alte, F., Hewitt, G., Sarek, G., Maslen, S.L., Tsutakawa, S.E., Borg, A., Kjcr, S., Tainer, J.A., et al. (2016). Mechanism and Regulation of DNA-Protein Crosslink Repair by the DNA-Dependent Metalloprotease SPRTN. Mol. Cell 64, 688-703.

Takimoto, C.H., and Arbuck, S.G. (1997). Clinical status and optimal use of topotecan. Oncology (Williston Park).

Vaz, B., Popovic, M., Newman, J.A., Fielden, J., Aitkenhead, H., Halder, S., Singh, A.N., Vendrell, I., Fischer, R., Torrecilla, I., et al. (2016). Metalloprotease SPRTN/DVC1 Orchestrates Replication-Coupled DNA-Protein Crosslink Repair. Mol. Cell 64, 704-719.

Vaz, B., Popovic, M., and Ramadan, K. (2017). DNA—Protein Crosslink Proteolysis Repair. Trends Biochem. Sci. 42, 483-495.

Yang, S.W., Burgin, A.B., Huizenga, B.N., Robertson, C.A., Yao, K.C., and Nash, H.A. (1996). A eukaryotic enzyme that can disjoin dead-end covalent complexes between DNA and type I topoisomerases. Proc. Natl. Acad. Sci. 93, 11534-11539.

1. A method for identifying a cancer that is predicted to respond to treatment with a Topoisomerase 1 (TOP1) inhibitor, the method comprising:
i. obtaining a sample of cancer cells/tissue from a subject; and
ii. detecting the expression of SPRTN enzyme and/or SPRTN mRNA in the sample of cancer cells/tissue;
wherein if the level of SPRTN enzyme and/or SPRTN mRNA in the sample is low then the subject is predicted to respond to treatment with a TOP1 inhibitor; or wherein if the level of SPRTN enzyme and/or SPRTN mRNA in the sample is high then the subject is predicted not to respond to treatment with a TOP1 inhibitor.

2. A method for identifying a subject with a metastatic colorectal cancer that is predicted to respond to treatment with a Topoisomerase 1 (TOP1) inhibitor, the method comprising:
i. obtaining a sample of primary colorectal cancer cells from the subject; and
ii. detecting the expression of SPRTN enzyme and/or SPRTN mRNA in the sample of primary cancer cells;
wherein if the level of SPRTN enzyme and/or SPRTN mRNA in the sample is low then the metastasis of the colorectal cancer is predicted to respond to treatment with a TOP1 inhibitor.

3. A method for identifying a subject with a metastatic colorectal cancer that is predicted not to respond to treatment with a Topoisomerase 1 (TOP1) inhibitor, the method comprising:
i. obtaining a sample of primary colorectal cancer cells from the subject; and
ii. detecting the expression of SPRTN enzyme and/or SPRTN mRNA in the sample of primary cancer cells;
wherein if the level of SPRTN enzyme and/or SPRTN mRNA in the sample is high then the metastatic cancer is predicted not to respond to treatment with a TOP1 inhibitor.

4. The method of any preceding claim wherein a low level of SPRTN enzyme expression is defined as that observed in a sample with an H-score of 100 or below.

5. The method of any preceding claim wherein a high level of SPRTN enzyme expression is defined as that observed in a sample with an H-score of above 100.

6. The method of any preceding claim wherein the subject has already been diagnosed with cancer.

7. The method of claim 6 wherein the subject has been diagnosed with colorectal cancer, optionally with primary colorectal cancer and/or metastatic colorectal cancer.

8. A method of stratifying patients into those which are expected to respond to therapy with a TOP1 inhibitor and those that are predicted not to respond to therapy with a TOP1 inhibitor or those predicted to respond poorly to therapy with a TOP1 inhibitor, the method comprising:
i. obtaining a sample of cancer cells/tissue from a patient; and
ii. detecting the expression of SPRTN enzyme and/or SPRTN mRNA in the sample of cancer cells/tissue;
wherein if the level of SPRTN enzyme and/or SPRTN mRNA in the sample is low then the subject is predicted to respond to treatment with a TOP1 inhibitor; or wherein if the level of SPRTN enzyme and/or SPRTN mRNA in the sample is high then the subject is predicted not to respond, or to respond poorly, to treatment with a TOP1 inhibitor.

9. The method of any preceding claim further comprising a step of:
iii. predicting that the subject, in particular a subject with a metastatic colorectal cancer, will respond to treatment with a TOP1 inhibitor if the level of SPRTN enzyme and/or SPRTN mRNA in the sample of cancer cells is low.

10. The method of any preceding claim further comprising a step of:
iii. predicting that the subject, in particular a subject with a metastatic colorectal cancer, will not respond to treatment with a TOP1 inhibitor if the level of SPRTN enzyme and/or SPRTN mRNA in the sample of cancer cells is high.

11. A kit for identifying a subject with a cancer that is predicted to respond to treatment with a TOP1 inhibitor, the kit comprising: detection means for detecting the SPRTN enzyme and/or SPRTN mRNA a sample of cancer cells from the subject; and instructions that if the level of SPRTN enzyme and/or SPRTN mRNA in the sample is low, then the subject is predicted to respond to treatment with a TOP1 inhibitor, or that if the level of SPRTN enzyme and/or SPRTN mRNA in the sample is high, then the subject is predicted not to respond to treatment with a TOP1 inhibitor.

12. A method of treating a cancer in a subject in need thereof, the method comprising:
iii. identifying a subject predicted to respond to therapy with a TOP1 inhibitor according to a method of any preceding claim; and
iv. administering a TOP1 inhibitor to the identified subject.

13. A method of treating a cancer in a subject, the method comprising administering a TOP1 inhibitor to a subject wherein the level of SPRTN and/or SPRTN mRNA in a sample of primary colorectal cancer cells from the subject is low, optionally the cancer to be treated is a metastatic colorectal cancer.

14. A TOP1 inhibitor for use in treating a cancer in a subject, wherein the subject has a low level of SPRTN enzyme and/or SPRTN mRNA in a sample of cancer cells from the subject.

15. The method of claim 14 wherein the sample of cells is a sample of primary colorectal cancer cells.

16. The method of claim 15 wherein the cancer to be treated is a metastatic colorectal cancer.

17. A method of selecting a cancer patient for treatment with a TOP1 inhibitor, the method comprising:
i. obtaining a sample of cancer cells from a cancer patient; and
ii. detecting SPRTN enzyme and/or SPRTN mRNA levels in the sample of cancer cells; and
iii. selecting the patient for treatment with a TOP1 inhibitor if the level of SPRTN enzyme and/or SPRTN mRNA in the sample is low.

18. A method of predicting if a cancer will respond to treatment with a TOP1 inhibitor, the method comprising:
i. obtaining a sample of cancer cells from a cancer patient; and
ii. detecting SPRTN enzyme and/or SPRTN mRNA expression in the sample of cancer cells; and
iii. predicting that if the level of SPRTN enzyme and/or SPRTN mRNA in the sample is low then the patient will respond to a TOP1 inhibitor.

19. The method or kit of any preceding claim wherein the TOP1 inhibitor is selected from the group comprising or consisting of camptothecin, an irinotecan, topotecan, lamellarin D, rubitecan, exatecan, bleotecan, 7-ethyl-10-hydroxycamptothecin (SN 38), derivatives based on camptothecin, and other DNA Topoisomerase 1 inhibitors.


SEQUENCE LISTING (14 sequences)

SEQ ID NO: 1 - DNA, 3734 bp, Homo sapiens
caaatgcgaacttaggctgttacacaactgctggggtctgttctcgccgcccgcccggcagtcaggcagcgtcgccgccgtggtagcagcctcagccgtttctggagtctcgggcccacagtcaccgccgcttacctgcgcctcctcgagcctccggagtccccgtccgcccgcacaggccggttcgccgtctgcgtctcccccacgccgcctcgcctgccgccgcgctcgtccctccgggccgacatgagtggggaccacctccacaacgattcccagatcgaagcggatttccgattgaatgattctcataaacacaaagataaacacaaagatcgagaacaccggcacaaagaacacaagaaggagaaggaccgggaaaagtccaagcatagcaacagtgaacataaagattctgaaaagaaacacaaagagaaggagaagaccaaacacaaagatggaagctcagaaaagcataaagacaaacataaagacagagacaaggaaaaacgaaaagaggaaaaggttcgagcctctggggatgcaaaaataaagaaggagaaggaaaatggcttctctagtccaccacaaattaaagatgaacctgaagatgatggctattttgttcctcctaaagaggatataaagccattaaagagacctcgagatgaggatgatgctgattataaacctaagaaaattaaaacagaagataccaagaaggagaagaaaagaaaactagaagaagaagaggatggtaaattgaaaaaacccaagaataaagataaagataaaaaagttcctgagccagataacaagaaaaagaagccgaagaaagaagaggaacagaagtggaaatggtgggaagaagagcgctatcctgaaggcatcaagtggaaattcctagaacataaaggtccagtatttgccccaccatatgagcctcttccagagaatgtcaagttttattatgatggtaaagtcatgaagctgagccccaaagcagaggaagtagctacgttctttgcaaaaatgctcgaccatgaatatactaccaaggaaatatttaggaaaaatttctttaaagactggagaaaggaaatgactaatgaagagaagaatattatcaccaacctaagcaaatgtgattttacccagatgagccagtatttcaaagcccagacggaagctcggaaacagatgagcaaggaagagaaactgaaaatcaaagaggagaatgaaaaattactgaaagaatatggattctgtattatggataaccacaaagagaggattgctaacttcaagatagagcctcctggacttttccgtggccgcggcaaccaccccaagatgggcatgctgaagagacgaatcatgcccgaggatataatcatcaactgtagcaaagatgccaaggttccttctcctcctccaggacataagtggaaagaagtccggcatgataacaaggttacttggctggtttcctggacagagaacatccaaggttccattaaatacatcatgcttaaccctagttcacgaatcaagggtgagaaggactggcagaaatacgagactgctcggcggctgaaaaaatgtgtggacaagatccggaaccagtatcgagaagactggaagtccaaagagatgaaagtccggcagagagctgtagccctgtacttcatcgacaagcttgctctgagagcaggcaatgaaaaggaggaaggagaaacagcggacactgtgggctgctgctcacttcgtgtggagcacatcaatctacacccagagttggatggtcaggaatatgtggtagagtttgacttcctcgggaaggactccatcagatactataacaaggtccctgttgagaaacgagtttttaagaacctacaactatttatggagaacaagcagcccgaggatgatctttttgatagactcaatactggtattctgaataagcatcttcaggatctcatggagggcttgacagccaaggtattccgtacatacaatgcctccatcacgctacagcagcagctaaaagaactgacagccccggatgagaacatcccagcgaagatcctttcttataaccgtgccaatcgagctgttgcaattctttgtaaccatcagagggcaccaccaaaaacttttgagaagtctatgatgaacttgcaaactaagattgatgccaagaaggaacagctagcagatgcccggagagacctgaaaagtgctaaggctgatgccaaggtcatgaaggatgcaaagacgaagaaggtagtagagtcaaagaagaaggctgttcagagactggaggaacagttgatgaagctggaagttcaagccacagaccgagaggaaaataaacagattgccctgggaacctccaaactcaattatctggaccctaggatcacagtggcttggtgcaagaagtggggtgtcccaattgagaagatttacaacaaaacccagcgggagaagtttgcctgggccattgacatggctgatgaagactatgagttttagccagtctcaagaggcagagttctgtgaagaggaacagtgtggtttgggaaagatggataaactgagcctcacttgccctcgtgcctgggggagagaggcagcaagtcttaacaaaccaacatctttgcgaaaagataaacctggagatattataagggagagctgagccagttgtcctatggacaacttatttaaaaatatttcagatatcaaaattctagctgtatgatttgttttgaattttgtttttattttcaagagggcaagtggatgggaatttgtcagcgttctaccaggcaaattcactgtttcactgaaatgtttggattctcttagctactgtatgcaaagtccgattatattggtgcgtttttacagttagggttttgcaataacttctatattttaatagaaataaattcctaaactcccttccctctctcccatttcaggaatttaaaattaagtagaacaaaaaacccagcgcacctgttagagtcgtcactctctattgtcatggggatcaattttcattaaacttgaagcagtcgtggctttggcagtgttttggttcagacacctgttcacagaaaaagcatgatgggaaaatatttcctgacttgagtgttcctttttaaatgtgaatttttatttctttttaattattttaaaatatttaaacctttttcttgatcttaaagatcgtgtagattggggttggggagggatgaagggcgagtgaatctaaggataatgaaataatcagtgactgaaaccattttcccatcatcctttgttctgagcattcgctgtaccctttaagatatccatctttttctttttaaccctaatctttcacttgaaagattttattgtataaaaagtttcacaggtcaataaacttagaggaaaatgagtatttggtccaaaaaaaggaaaaataatcaagattttagggcttttattttttcttttgtaattgtgtaaaaaatggaaaaaaacataaaaagcagaattttaatgtgaagacattttttgctataatcattagttttagaggcattgttagtttagtgtgtgtgcagagtccatttcccacatctttcctcaagtatcttctatttttatcatgaattcccttttaatcaactgtaggttatttaaaataaattcctacaacttaatggaaa

SEQ ID NO: 2 - protein, 765 aa, Homo sapiens
MSGDHLHNDSQIEADFRLNDSHKHKDKHKDREHRHKEHKKEKDREKSKHSNSEHKDSEKKHKEKEKTKHKDGSSEKHKDKHKDRDKEKRKEEKVRASGDAKIKKEKENGFSSPPQIKDEPEDDGYFVPPKEDIKPLKRPRDEDDADYKPKKIKTEDTKKEKKRKLEEEEDGKLKKPKNKDKDKKVPEPDNKKKKPKKEEEQKWKWWEEERYPEGIKWKFLEHKGPVFAPPYEPLPENVKFYYDGKVMKLSPKAEEVATFFAKMLDHEYTTKEIFRKNFFKDWRKEMTNEEKNIITNLSKCDFTQMSQYFKAQTEARKQMSKEEKLKIKEENEKLLKEYGFCIMDNHKERIANFKIEPPGLFRGRGNHPKMGMLKRRIMPEDIIINCSKDAKVPSPPPGHKWKEVRHDNKVTWLVSWTENIQGSIKYIMLNPSSRIKGEKDWQKYETARRLKKCVDKIRNQYREDWKSKEMKVRQRAVALYFIDKLALRAGNEKEEGETADTVGCCSLRVEHINLHPELDGQEYVVEFDFLGKDSIRYYNKVPVEKRVFKNLQLFMENKQPEDDLFDRLNTGILNKHLQDLMEGLTAKVFRTYNASITLQQQLKELTAPDENIPAKILSYNRANRAVAILCNHQRAPPKTFEKSMMNLQTKIDAKKEQLADARRDLKSAKADAKVMKDAKTKKVVESKKKAVQRLEEQLMKLEVQATDREENKQIALGTSKLNYLDPRITVAWCKKWGVPIEKIYNKTQREKFAWAIDMADEDYEF

SEQ ID NO: 3 - DNA, 3223 bp, Homo sapiens
agtcctggtcctcggctaggcggcttggggtcgcggcgtaactggggagccagcctgacgccggcggaccccgcctgtgatcctggcaacgatggatgatgacttgatgttggcactgcggcttcaggaggagtggaacttgcaggaggcggagcgcgatcatgcccaggagtccctgtcgctagtggacgcgtcgtgggagttggtggaccccacaccggacttgcaggcactgtttgttcagtttaacgaccaattcttctggggccagctggaggccgtcgaggtgaagtggagcgtgcgaatgaccctgtgtgctgggatatgcagctatgaagggaagggtggaatgtgttccatccgtctcagcgaaccccttttgaagttgaggccaagaaaggatcttgtagagaccctcctgcatgaaatgatacatgcctatttatttgtcactaataacgacaaagaccgagaagggcatggtccagaattttgtaaacatatgcatcgcatcaacagcctgactggagccaatataacggtataccatacttttcacgatgaggtggatgagtatcggcgacactggtggcgctgcaatgggccgtgccagcacaggccaccgtattacggctatgtcaaacgagctactaacagggaaccctctgctcatgactattggtgggctgagcaccagaaaacctgtggaggcacttacataaaaatcaaggaaccagagaattactcaaaaaaaggcaaaggaaaggcaaaactaggaaaggaaccagtattggccgcagagaataaagataaacccaacagaggtgaggcccagctagtaatcccttttagtgggaaaggatatgttctaggagaaacaagcaatttaccttcacctgggaaactgatcacttcacatgccattaataaaacccaagatcttttaaatcaaaaccattcagcaaatgctgtaagacctaattctaaaatcaaggtgaaatttgaacagaatggttcaagtaaaaattctcatctggtctcccctgctgttagtaacagtcaccaaaatgttctaagcaactactttcctagagtatcatttgccaaccaaaaggctttcagaggtgtgaatggatctccaaggataagtgtaacagttggcaacatccctaaaaactcagtctcttctagttctcagagaagggtttcatcttctaagatatccctaagaaattcttcaaaagtaacggaatcagcatctgtgatgccatcccaggatgtgagtgggtctgaagatacattcccaaataaacgacctaggctagaagataagactgtttttgacaatttttttatcaagaaagagcaaataaaaagcagtggtaatgatccaaagtatagtacaaccacagctcagaattccagcagttcatccagtcagagcaaaatggttaattgcccagtttgtcagaatgaagttctggagtctcagattaatgagcacttggactggtgccttgaaggtgacagcatcaaagtcaaaagcgaagaaagtctttgaaaaaggtttcaaagtctcaagtaccacctgtattatctcactaatgtgctatgtcagccagtcaggaagttctggttaatactaagatttgtaggttataatctagttcacataaccaatagaaagtgtcctattttatatatacgcatataagattgtaattttaagatgttttgtgtctcagggtgctacattcactcttgccttaggtatactgtaacccaggttctgcctgtcgtgtataagttttagatacttttgttctttcttgctcttaaggattttaaaaacctgttaatctttttatttgtatactttcctaaaaatattcatatggggaatcctgtcaggtgtttggttatattgactatttattaatagtattagaactcattccctgaactgatgtaaatcttcatagtgtcagacatactgaccaaaaccacaatctagactacaaagtatattgttttagagtactcaaattgtattatttattaatttttttgtttgcaaaatcttaacaggaactgtattttctatattttaaagaattttatttgtcccacttttactaaacagtggcagcagattttaagttaaagaatatggaatatagtaaaataagtaaatttcttttggaatatttttagtaacaaatagccactataattctgtaggccaaattttatattgagtttagctgttttctcaaaatttagcagagtggttaaaattctgtgctgataagtaactgatacatataacataaacataacaaagttgcctagttgatgtaacagtggaaagttatctggaaatagtattttgaactttaagccaagtttaaaacattataataaaaggaataccatttgtgcattttaagtaatcttttttaaaaaaaatattttccatgttatagggaaaggacaaagagacttttatcagtttgctttttgtcttgtggctgtacatgctgttggcatagccctaacacagttgttcacaagttttcttttttcttgttgcaattttccttcactttgttgtaatacaggtgcacaaatcttaagtgcacagctgggtaaacttttacagtgttcacctgtgtaactaccacccggatcaagttagagaacacttccattgccacagaaggcttcctataggtgtctgttcccagttgatacccatgaccctcaccacctccagaggtccccactgttttcaccccatcgtcgcagattattttttaattttataatgtagtatctttgttcctatgtatagcaggagttcattttcattgctcttgcatttgtatgaatatactagaatttattcatccattctaatgttaatggacatttgaattatttccagtttggggctgttaagcataatgctgctaagaacattcttatcttttctgttgggtatatatacaggaatggaattgctgagtcacggggtatatttctgtttagctttagtaaattctgccagtttacactcctaccagcaatgtatgagagttttagttgttcaccattcataacactgttagcagcccttttcgttttagactggttggctgttcacaaattttgatattccttaaagcattaaaacattttaataaatacttataaataggta

SEQ ID NO: 4 - protein, 489 aa, Homo sapiens
MDDDLMLALRLQEEWNLQEAERDHAQESLSLVDASWELVDPTPDLQALFVQFNDQFFWGQLEAVEVKWSVRMTLCAGICSYEGKGGMCSIRLSEPLLKLRPRKDLVETLLHEMIHAYLFVTNNDKDREGHGPEFCKHMHRINSLTGANITVYHTFHDEVDEYRRHWWRCNGPCQHRPPYYGYVKRATNREPSAHDYWWAEHQKTCGGTYIKIKEPENYSKKGKGKAKLGKEPVLAAENKDKPNRGEAQLVIPFSGKGYVLGETSNLPSPGKLITSHAINKTQDLLNQNHSANAVRPNSKIKVKFEQNGSSKNSHLVSPAVSNSHQNVLSNYFPRVSFANQKAFRGVNGSPRISVTVGNIPKNSVSSSSQRRVSSSKISLRNSSKVTESASVMPSQDVSGSEDTFPNKRPRLEDKTVFDNFFIKKEQIKSSGNDPKYSTTTAQNSSSSSSQSKMVNCPVCQNEVLESQINEHLDWCLEGDSIKVKSEESL

SEQ ID NO: 5 - RNA, 19 nt, Homo sapiens
cguacgcggaauacuucga

SEQ ID NO: 6 - RNA, 21 nt, Homo sapiens
aaguaggguaugaugacauug

SEQ ID NO: 7 - RNA, 21 nt, Homo sapiens
aagauggaucucauugaccua

SEQ ID NO: 8 - RNA, 21 nt, Homo sapiens
aacagccauucucaaacagaa

SEQ ID NO: 9 - DNA, 21 bp, Homo sapiens
ctcatcgacctctaccagaaa

SEQ ID NO: 10 - DNA, 21 bp, Homo sapiens
cggctggagatctaccaggaa

SEQ ID NO: 11 - RNA, 19 nt, Homo sapiens
cuuccaggacccagaauaa

SEQ ID NO: 12 - RNA, 21 nt, Homo sapiens
cacgaugagguggaugaguau

SEQ ID NO: 13 - RNA, 19 nt, Homo sapiens
ggucccuguugagaaacga

SEQ ID NO: 14 - RNA, 19 nt, Homo sapiens
ggauauugcguuuggaaca